(19) United States
(12) Patent Application Publication
(10) Pub. No.: US 2023/0358729 A1
(43) Pub. Date: Nov. 9, 2023

(54) DERIVATION OF TROPHOBLAST ORGANOIDS FROM NAIVE HUMAN PLURIPOTENT STEM CELLS

(71) Applicant: Washington University, St. Louis, MO (US)

(72) Inventors: Thorold Theunissen, St. Louis, MO (US); Rowan Karvas, St. Louis, MO (US)

(73) Assignee: Washington University, St. Louis, MO (US)

(21) Appl. No.: 18/312,488

(22) Filed: May 4, 2023

Related U.S. Application Data
(60) Provisional application No. 63/338,270, filed on May 4, 2022.

(51) Int. Cl. G01N 33/50; C12N 5/073; C12Q 1/6869; C12Q 1/70

(52) U.S. Cl. CPC G01N 33/5082; C12N 5/0605; C12Q 1/6869; C12Q 1/701; C12N 2513/00; C12Q 2600/158; C12Q 2600/156; C12N 2533/90

(57) ABSTRACT

A 3D model system of human placental development that includes 3D stem cell-derived trophoblast organoids comprising self-organized trophoblast stem cells isolated from naive human pluripotent stem cells, and uses thereof, are described.

### CROSS-REFERENCE TO RELATED APPLICATIONS

This application claims priority from U.S. Provisional Application Ser. No. 63/338,270 filed on May 4, 2022, which is incorporated herein by reference in its entirety.

### STATEMENT REGARDING FEDERALLY SPONSORED RESEARCH OR DEVELOPMENT

This invention was made with government support under GM137418 and EB028092 awarded by the National Institutes of Health. The government has certain rights in the invention.

### MATERIAL INCORPORATED-BY-REFERENCE

Not applicable.

### FIELD OF THE INVENTION

The present disclosure generally relates to systems and methods for trophoblast organoids from naive human pluripotent stem cells.

### BACKGROUND OF THE INVENTION

Trophoblast organoids derived from placental villi provide a 3D model system of human placental development, but access to first-trimester tissues is limited. Trophoblast organoids serve as a useful in vitro model system of the human placenta, but have thus far only been isolated from first-trimester placental tissues, which are inaccessible to many researchers for ethical and practical reasons.

### SUMMARY OF THE INVENTION

Among the various aspects of the present disclosure is the provision of systems and methods for trophoblast organoids from naïve human pluripotent stem cells.

Briefly, therefore, the present disclosure is directed to systems and methods related to trophoblast organoids from human pluripotent stem cells.

In one aspect, a model system of human placental development comprising a 3D stem cell-derived trophoblast organoid (SC-TO) comprising self-organized trophoblast stem cells is disclosed, wherein the trophoblast stem cells are isolated from naïve human pluripotent stem cells. In some aspects, the self-organized trophoblast stem cells of the SC-TO comprise a portion of cytotrophoblast cells positioned at a periphery of the SC-TO and a portion of multinucleated syncytiotrophoblast cells positioned within an interior of the SC-TO. In some aspects, the self-organized trophoblast stem cells of the SC-TO further comprise a portion of primitive extravillous trophoblasts. In some aspects, the SC-TO displays clonal X chromosome inactivation patterns. In some aspects, the SC-TO exhibits vulnerability to a pathogen selected from SARS-CoV-2 or Zika virus. In some aspects, the vulnerability is correlated with an expression level of entry factors of the pathogens. In some aspects, the entry factors comprise at least one of ACE2, TMPRSS2, and TYRO3. In some aspects, the system also includes a 3D extravillous trophoblast organoid comprising a plurality of extravillous trophoblasts produced by differentiating the self-organized trophoblast stem cells into the extravillous trophoblasts.

In another aspect, a method of generating a stem cell-derived trophoblast organoid (SC-TO) is disclosed. The organoid includes self-organized trophoblast stem cells isolated from naïve human pluripotent stem cells. The method includes isolating naïve human pluripotent stem cells from human samples, seeding the naïve human pluripotent stem cells in a 3D scaffold, and culturing the naïve human pluripotent stem cells in a TSC culture composition. In some aspects, the self-organized trophoblast stem cells of the SC-TO comprise a portion of cytotrophoblast cells positioned at a periphery of the SC-TO and a portion of multinucleated syncytiotrophoblast cells positioned within an interior of the SC-TO. In some aspects, the self-organized trophoblast stem cells of the SC-TO further comprise a portion of primitive extravillous trophoblasts. In some aspects, the SC-TO displays clonal X chromosome inactivation patterns. In some aspects, the SC-TO exhibits vulnerability to a pathogen selected from SARS-CoV-2 or Zika virus. In some aspects, the vulnerability is correlated with an expression level of entry factors of the pathogens. In some aspects, the entry factors comprise at least one of ACE2, TMPRSS2, and TYRO3.

In an additional aspect, a method of screening for molecules as a treatment for a pregnancy-related disease, complication, or disorder is disclosed that includes inducing the pregnancy-related disease, complication, or disorder in a 3D model system of human placental development, and administering a candidate molecule to the SC-TO and selecting the candidate molecule as a treatment if the candidate molecule reduces the pregnancy-related disease, complication, or disorder in the SC-TO. The system includes a 3D stem cell-derived trophoblast organoid (SC-TO) comprising self-organized trophoblast stem cells isolated from naïve human pluripotent stem cells. In some aspects, the pregnancy-related disease, complication, or disorder comprises a viral infection. In some aspects, the viral infection comprises an infection by a pathogen selected from a SARS-CoV-2 virus and a Zika virus. In some aspects, the at least a portion of the self-organized trophoblast stem cells of the SC-TO expresses at least one entry factor of the pathogen. In some aspects, the at least one entry factor is selected from ACE2, TMPRSS2, and TYRO3.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic illustration of SC-TO derivation. Human trophoblast stem cells (hTSCs) were sourced from naïve human pluripotent stem cells (hPSCs), human blastocyst outgrowths, or primary cytotrophoblasts (CTBs) of first-trimester placentas. hTSCs were maintained in an hTSC medium and transferred to 3D culture in a Matrigel droplet and maintained in trophoblast organoid medium (TOM).

FIG. 1B contains a series of brightfield images of H9 and CT30 SC-TOs. Both SC-TO lines were maintained for 10 passages and exhibited a similar overall structure. Organoid morphology consists of opaque, largely CTB, with some interior STB (red arrows) and heavily syncytialized, clear cystic STB (blue asterisks). Stereoscopic images (left, middle columns) scale=1 mm. Widefield images (right) scale=200 μm.

FIG. 1C contains a series of graphs of changes in expression levels (fold change) of various genes obtained using quantitative gene expression analysis during derivation of SC-TOs from naïve hPSCs. Primed hPSCs expressed VIM and ZIC2, naïve hPSCs expressed DNMT3L and KLF17, and trophoblast cells in 2D hTSC culture and 3D SC-TOs expressed GATA3 and TFAP2C. Fold change is plotted relative to H9 5i/L/A. Error bars indicate mean±SD of at least 2 biological replicates. *p value<0.0001.

FIG. 1D is a series of graphs of changes in expression levels (fold change) of various genes obtained using quantitative gene expression analysis for chromosome 19 microRNAs in hTSCs and SC-TOs compared to primed and naïve stem cells. Fold change is plotted relative to H9 mTeSR1. Error bars indicate mean±SD of at least 2 biological replicates. ****p<0.0001.

FIG. 1E is a set of immunofluorescence images of trophoblast markers in H9 SC-TOs. All images represent single sections of confocal imaging analysis for the following markers: CDH1, GATA3 (top); TP63 and MKI67 (middle); and KRT7 (bottom).

FIG. 1F is a set of light sheet microscopic images of a representative CT30 SC-TO. Organoids were stained for epithelial CTB marker CDH1 and STB marker SDC1 followed by optical clearing. The bottom image shows the 3D volume rendered in Amira software.

FIG. 1G is a set of light sheet microscopic images of a representative H9 SC-TO. Organoids were stained for epithelial CTB marker CDH1 and STB marker SDC1 followed by optical clearing. The bottom image shows the 3D volume rendered in Amira software.

FIG. 1H is a graph of the quantification of signals from whole 3D organoids. CT30: n=19 SC-TOs, H9: n=14 SC-TOs. The volumes measured were DAPI, SDC1, and CDH1. No significant differences were observed between H9 and CT30 SC-TO for any of the examined proteins.

FIG. 1I is a graph of the quantification of hCG ELISA analysis of secreted hCG levels in cell culture media of primed hPSCs (negative control), naïve hPSCs, and SC-TOs. This experiment was performed on SC-TOs generated from five independent hTSC lines. Paired t-test: ** p value<0.01.

FIG. 1J is a flow cytometry analysis plot for HLA-ABC (W6/32) in H9 5i/L/A naïve hPSCs (negative control), H9 mTeSR1 primed hPSCs (positive control), H9 hTSCs and SC-TOs, and CT30 hTSCs and SC-TOs.

FIG. 2A is a UMAP plot of the cellular composition of SC-TOs revealed by single-cell RNA-sequencing (scRNA-seq). Uniform Manifold Approximation and Projection (UMAP) subclusters include CTB-1 and CTB-2, STB-1 and STB-2, and a small primitive EVT population. These studies were performed on two replicates of SC-TOs generated from two independent genetic backgrounds: H9 naïve hTSCs and CT30 primary hTSCs.

FIG. 2B is a pair of plots of an in silico analysis of SC-TO differentiation patterns. Pseudotime analyses indicate the relatedness of subclusters between CT30 and H9 SC-TOs, allowing inference of two predominant differentiation trajectories, both of which emerge from CTB-1 (red arrows).

FIG. 2C is a pair of dot plots indicating the expression of trophoblast progenitor and lineage markers in five distinct clusters as shown in FIG. 2A. Average gene expression levels and the percentage of cells that express each gene are presented with differential color intensities and circle sizes, respectively.

FIG. 2D is a set of UMAP plots indicating the expression of the CTB markers CDH1 and TEAD4 in H9 and CT30 SC-TOs.

FIG. 2E is a set of UMAP plots indicating the expression of the STB markers CGA and ERVW-1 in H9 and CT30 SC-TOs.

FIG. 2F is a set of UMAP plots indicating expression of the EVT markers ITGA2 and HLAG in H9 and CT30 SC-TOs.

FIG. 2G is a pair of dot plots indicating the expression of placenta-specific imprinted genes in H9 and CT30 SC-TOs. Average gene expression levels and the percentage of cells that express each gene are presented with differential color intensities and circle sizes, respectively.

FIG. 2H is a plot of the integration of SC-TO UMAP data with scRNA-seq analysis of human post-implantation stage trophoblasts. These data represent combined scRNA-seq data from two independent SC-TO lines (H9 and CT30).

FIG. 2I is a plot similar to FIG. 2H except that trophoblast subpopulations are highlighted and separated by embryonic day (E7-14).

FIG. 2J is a plot of the integration of SC-TO UMAP data with scRNA-seq analysis containing trophoblast cells from primary human placental tissues. SC-TO clusters are highlighted in colors.

FIG. 2K is a plot similar to FIG. 2J except that primary placental samples are highlighted in colors. See also FIGS. 7 and 8.

FIG. 3A is a graph of an allele-specific gene expression analysis using SNPs located within transcribed regions of X-linked genes in primed H9 hESCs in mTeSR1. Allelic frequencies were analyzed for those SNPs covered by at least 10 reads in naïve hESCs. Asterisks mark genes reported to escape X inactivation.

FIG. 3B is a graph of an allele-specific gene expression analysis using SNPs located within transcribed regions of X-linked genes in naïve hESCs that were derived in 5i/L/A. Allelic frequencies were analyzed for those SNPs covered by at least 10 reads in naïve hESCs. Asterisks mark genes reported to escape X inactivation.

FIG. 3C is a UMAP plot that denotes the identities of H9 SC-TO clusters in 10× single-cell transcriptome data, which were used to calculate allelic frequencies of X-linked genes in FIGS. 3D-E and 8A.

FIG. 3D is a UMAP plot indicating allele-specific expression of DMD in SC-TOs derived from H9 naïve hESCs. Cells expressing the reference allele are indicated in teal, cells expressing the alternative allele are indicated in navy, and the few cells expressing both alleles are indicated in blue.

FIG. 3E is a UMAP plot indicating allele-specific expression of NRK in SC-TOs derived from H9 naïve hESCs. Cells expressing the reference allele are indicated in teal and cells expressing the alternative allele are indicated in navy.

FIG. 3F is a schematic (first sheet) and representative phase and fluorescence images (second sheet) of XCI dynamics during SC-TO derivation from naïve hESCs, as revealed using WIBR3 hESCs carrying a dual fluorescent reporter in both alleles of the X-linked MECP2 locus. PXGGY/A: alternative naïve hPSC induction medium; SAVECY: hTSC medium; TOM: trophoblast organoid medium.

FIG. 3G is a set of flow cytometry analysis plots for MECP2-GFP and MECP2-tdTomato on samples shown in FIG. 3F. FACS plots are representative of 2 independent biological replicates. Tables (right) indicate the mean percentage of cells within each quarter and the standard deviation. See also FIG. 9.

FIG. 4A is a schematic representation of signaling requirements to maintain SC-TOs or induce differentiation towards specialized 3D SC-EVTOs.

FIG. 4B is a set of phase contrast views of SC-TOs maintained in trophoblast organoid medium (TOM), which promotes a smooth and spherical structure. Both CT30 and H9 SC-TOs differentiated into 3D SC-EVTOs exhibit migratory EVTs, as indicated by red arrows. The scale bar depicts 200 μm.

FIG. 4C is a graph of an ELISA analysis of secreted MMP2 from SC-TO lines exposed to EVT-promoting media. These studies represent two biological replicate experiments (H9 and CT30 SC-TOs). Error bars indicate mean±1 SE of three technical replicates. *p-value<0.01.

FIG. 4D is a set of graphs of quantitative gene expression analysis of general trophoblast markers ELF5 and TEAD4, the STB differentiation marker ERVW1, and EVT differentiation markers HLAG, MMP2, and FN1 upon differentiation of H9 and CT30 SC-TOs into SC-EVTOs. Fold change is plotted relative to H9 SC-TO. Error bars indicate mean±1 SD of 2-3 biological replicates.

FIG. 4E is a set of maximal projection images of SC-EVTOs that demonstrate overlapping HLAG and MMP2 expression. These data represent differentiation experiments performed with three independent SC-TO lines (H9, CT30, and BT5).

FIG. 4F is a schematic of the SC-TO/SC-EVTO invasion assay to test interactions with immortalized human endometrial fibroblasts and glandular epithelial cells embedded in a 3D Matrigel matrix. H9 SC-TOs were lentivirally labeled with a constitutive GFP vector to enable the quantification of invasive projections.

FIG. 4G is a graph of the quantification of SC-TO/SC-EVTO invasive projections between days 1 and 7 of co-culture with or without human endometrial cells.

FIG. 4H is a set of representative images of SC-TO/SC-EVTO invasion assay in the presence (+ENDO) or absence (−ENDO) of human endometrial cells. The most pronounced invasive projections were observed using SC-EVTOs in the presence of endometrial cells. GFP constitutively marks SC-TOs/SC-EVTOs. ITGA5B1 staining indicates invasive EVTs. Scale bar=100 μm.

FIG. 5A is a pair of UMAP plots indicating the expression of the SARS-CoV-2 entry factors ACE2 and TMPRSS2. These data represent combined scRNA-seq data from two H9 SC-TO replicates.

FIG. 5B is a pair of UMAP plots indicating the expression of the ZIKV entry factors TYRO3 and MERTK. These data represent combined scRNA-seq data from two H9 SC-TO replicates.

FIG. 5C is a schematic of VSV-eGFP-Glycoprotein (VSV-G) and VSV-eGFP-SARS-CoV-2-Spike (VSV-S) infections of SC-TOs. The presence of virally encoded GFP was assayed by flow cytometry and fluorescent microscopy.

FIG. 5D is a set of images of infection of H9 SC-TOs with VSV-G that showed widespread infection. These data are representative of two biological replicates. The scale bar depicts 200 μm.

FIG. 5E is a set of images of VSV-S infection of SC-TOs that demonstrated more limited infection compared to VSV-G. Yellow arrows indicate the sparse CTBs infected while the red arrow identifies the GFP-positive multinucleated STB. These data are representative of two biological replicates. The scale bar depicts 200 μm.

FIG. 5F is a pair of flow cytometry analysis plots for virally encoded GFP in dissociated H9 SC-TOs following infection with VSV-S or VSV-G.

FIG. 5G is a schematic and a set of confocal IF images wherein SC-TOs were infected with a clinical isolate of live SARS-CoV-2 (MOI=3). Single-plane confocal IF imaging for the SARS-CoV-2 Spike protein revealed no significant difference between infected vs. uninfected SC-TOs. Furthermore, morphological signs of infection were absent. These experiments were performed on two independent SC-TO lines (H9 and CT30). The scale bar depicts 100 μm.

FIG. 5H is a schematic and a pair of images wherein H9 SC-TOs were infected with the Brazilian strain of ZIKV and assessed by IF for the capsid envelope protein. 3D view of SC-TO (top) or SC-EVTO (bottom) showed widespread infection among all organoid cell types. The scale bar depicts 200 μm. See also FIG. 10.

FIG. 6A is a set of images of SC-TO lines obtained from H9, WIBR3, CT30, and BT5 hTSCs, which were derived from naïve hPSCs (H9, WIBR3), primary trophoblast (CT30), and a human blastocyst (BT5), respectively, all showed similar overall organoid morphology by phase contrast imaging over time in TOM medium. The scale bar depicts 400 μm.

FIG. 6B is a set of stereoscopic images that demonstrated that SC-TO lines H9, CT30, and BT5 are capable of long-term culture over 8-10 passages in TOM medium. The scale bar depicts 1000 μm.

FIG. 6C is a graph of quantitative gene expression analysis for trophoblast marker ELF5 during derivation of SC-TOs from naïve hPSCs. Fold change is plotted relative to H9 5i/L/A. Error bars indicate mean±SD of at least 2 biological replicates. *p value<0.01.

FIG. 6D is a set of images with immunofluorescence analysis that reveals that primary hTSC-derived SC-TOs express trophoblast markers TP63, GATA3, CDH1, and KRT7 similarly to H9 SC-TOs (see FIG. 1E). Proliferative signal MKI67 overlaps strongly with CDH1+ cells on the periphery of the organoids. The maximal projection is shown for BT5. Scale bar=100 μm unless otherwise noted.

FIG. 6E is a pair of images with immunofluorescence analysis that reveals that CGA is ubiquitously expressed in a subset of entirely syncytialized organoids within CT30 and H9 SC-TO cultures. Scale bar=100 μm.

FIG. 6F is a photo of pregnancy test strips that were used to test for secreted hCG in SC-TO cell cultures (CT30, H9, and WIBR3).

FIG. 7A is a set of plots of differential gene expression analyses that indicate few significant changes in gene expression between matched clusters in H9 and CT30 SC-TOs. Blue dots indicate individual genes that are differentially expressed (p-value<0.05).

FIG. 7B is a pair of pie charts of the relative proportions of the various subpopulations within H9 and CT30 SC-TOs based on scRNA-seq data.

FIG. 7C is a pair of volcano plots indicating differentially expressed genes (DEGs) between subpopulations CTB-1 and CTB-2 (first sheet) and STB-1 and STB-2 (second sheet). These data represent combined scRNA-seq data from two independent SC-TO lines (H9 and CT30) indicating the top DEGs ranked according to adjusted P value (Wilcoxon signed-rank test; BH correction).

FIG. 7D is a set of UMAP plots indicating the fraction of G2/M-phase-associated genes that mark actively dividing cells (second sheet) and S-phase-associated genes (third sheet) expressed in SC-TOs. These data represent combined scRNA-seq data from two independent SC-TO lines (H9 and CT30).

FIG. 8A is a set of plots of Gene Ontology (GO) analyses indicating significantly enriched terms in the five distinct SC-TO subpopulations. These data represent combined scRNA-seq data from two independent SC-TO lines (H9 and CT30).

FIG. 8B is a heatmap of differentially expressed genes (DEGs) in the five distinct SC-TO subpopulations. These data represent combined scRNA-seq data from two independent SC-TO lines (H9 and CT30).

FIG. 9A is a set of graphs of allele-specific expression of X-linked genes based on 10× single-cell transcriptome data of SC-TOs generated from H9 naïve hESCs. None of the examined SNP-containing transcripts showed substantial bi-allelic expression, except for PLCXD1, which is known to escape XCI (Balaton et al., 2015). Only SNPs that were covered by at least 50 reads over all single cells were considered in this analysis.

FIG. 9B is a set of phase and fluorescent images of WIBR3 MECP2-GFP/tdTomato naïve hESCs derived in 5i/L/A and PXGGY/A for 2 passages.

FIG. 9C is a pair of flow cytometry analysis plots that indicate a fraction (15.5%) of cells that retain mono-allelic MECP2 expression in 5i/L/A conditions. In contrast, nearly all cells displayed bi-allelic MECP2 expression in PXGGY/A.

FIG. 9D is a pie chart of the percentage of individual organoids that exclusively expressed GFP, tdTomato, or a mixture of both signals upon SC-TO derivation from WIBR3 MECP2-GFP/tdTomato naïve hESCs derived in PXGGY/A (n=201 SC-TOs counted).

FIG. 10A is a set of images wherein CT30 SC-TOs demonstrated a similar infection pattern by VSV pseudoviruses as H9 SC-TOs (see FIG. 5D-E). VSV-eGFP-G (VSV-G) showed robust infection while little to no infection was seen in CT30 SC-TOs treated with the VSV-eGFP-SARS-CoV-2-Spike (VSV-S) pseudovirus. Scale bar indicates 200 μm.

FIG. 10B is a set of images wherein a subset of STBs within H9 SC-TOs was infected with the VSV-S pseudovirus. Shown here are three protruding syncytial areas, marked by a positive CGB signal and a lack of CDH1. The red arrow indicates a GFP+ STB area that has become infected, while the green arrows indicate protruding STBs that were not infected, indicating that a subset of STBs within SC-TOs may be susceptible to SARS-CoV-2 entry.

FIG. 10C is a set of flow cytometry analysis plots of virally encoded GFP in 2D hTSCs following infection with VSV-G or VSV-S. This experiment was performed with 3 biological replicates on two independent 2D hTSC lines (CT30 and H9).

FIG. 10D is a pair of fluorescence images of 2D STBs generated from CT30 hTSCs that were uninfected. 24 well plates were stained with Phalloidin (magenta) to indicate total cell density. Yellow arrows indicate GFP-positive cell clusters. Total cell density is indicated by Phalloidin staining (magenta).

FIG. 10E is a pair of fluorescence images of 2D STBs generated from CT30 hTSCs that were infected with the VSV-S pseudovirus. 24 well plates were stained with Phalloidin (magenta) to indicate total cell density. Yellow arrows indicate GFP-positive cell clusters. Total cell density is indicated by Phalloidin staining (magenta).

FIG. 10F is a pair of fluorescence images of 2D STBs generated from CT30 hTSCs that were infected with the VSV-G. 24 well plates were stained with Phalloidin (magenta) to indicate total cell density. Yellow arrows indicate GFP-positive cell clusters. Total cell density is indicated by Phalloidin staining (magenta).

FIG. 10G is a pair of images of the magnified region of VSV-G and VSV-S infection of multinucleated 2D STBs derived from CT30 hTSCs. Cells are co-stained with DAPI (blue) and Phalloidin (magenta). The lower image shows a subset of STBs that was infected by VSV-S. Scale bar=100 μm.

Those of skill in the art will understand that the drawings, described below, are for illustrative purposes only. The drawings are not intended to limit the scope of the present teachings in any way.

### DETAILED DESCRIPTION OF THE INVENTION

The present disclosure is based, at least in part, on the discovery that trophoblast stem cells isolated from naïve human pluripotent stem cells (hPSCs) can efficiently self-organize into 3D stem cell-derived trophoblast organoids (SC-TOs) with a villous architecture similar to primary trophoblast organoids. As shown herein, compositions and methods are described to generate trophoblast organoids from naïve hPSCs that can provide an accessible 3D model system of the developing placenta and its susceptibility to emerging pathogens.

One aspect of the disclosure describes that single-cell transcriptome analysis reveals the presence of distinct cytotrophoblast and syncytiotrophoblast clusters and a small cluster of extravillous trophoblasts, which can closely correspond to trophoblast identities in the post-implantation embryo. In some embodiments, organoid cultures display clonal X chromosome inactivation patterns previously described in the human placenta. In some embodiments, it is demonstrated that SC-TOs exhibit selective vulnerability to emerging pathogens (SARS-CoV-2 and Zika virus). In some embodiments, this can correlate with expression levels of respective entry factors. In some embodiments, the generation of trophoblast organoids from naïve hPSCs provides an accessible 3D model system of the developing placenta and its susceptibility to emerging pathogens.

In some embodiments, an efficient method for generating 3D trophoblast organoids (also known as “mini-placentas”) from naive human pluripotent stem cells (hPSCS) is described. In some embodiments, trophoblast organoids can serve as a useful in vitro model system of the human placenta. In some embodiments, the isolation of stem-cell-derived trophoblast organoids (SC-TOs) facilitates disease modeling and drug screening for pregnancy-related complications.

Screening

In some aspects, the disclosed 3D model systems of human placental development may be used to implement various methods for screening treatments for a pregnancy-related disease, complication, or disorder.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to stem cells, growth factors, and a 3D scaffold. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Stem Cell-Derived Trophoblast Organoids Model Human Placental Development and Susceptibility to Emerging Pathogens

In this example, it is described that stem cell-derived trophoblast organoids model human placental development and susceptibility to emerging pathogens. 3D trophoblast organoids derived from naïve and primary hTSCs displayed comparable tissue architecture, placental hormone secretion, and capacity for long-term self-renewal. Single-cell transcriptomes revealed the cellular complexity of 3D organoids generated from naïve and primary hTSCs, and SC-TO subpopulations corresponded to trophoblast identities in the human post-implantation. XCI dynamics were described during trophoblast organoid generation from naïve hPSCs. Further, the differentiation of SC-TOs into invasive 3D EVT organoids occurred, which displayed selective vulnerability to emerging viral pathogens.

### ABSTRACT

Trophoblast organoids derived from placental villi provide a 3D model system of human placental development, but access to first-trimester tissues is limited. Here it is reported that trophoblast stem cells isolated from naïve human pluripotent stem cells (hPSCs) can efficiently self-organize into 3D stem cell-derived trophoblast organoids (SC-TOs) with a villous architecture similar to primary trophoblast organoids. Single-cell transcriptome analysis reveals the presence of distinct cytotrophoblast and syncytiotrophoblast clusters and a small cluster of extravillous trophoblasts, which closely correspond to trophoblast identities in the post-implantation embryo. These organoid cultures display clonal X chromosome inactivation patterns previously described in the human placenta. It is further demonstrated that SC-TOs exhibit selective vulnerability to emerging pathogens (SARS-CoV-2 and Zika virus), which correlates with expression levels of their respective entry factors. The generation of trophoblast organoids from naïve hPSCs provides an accessible 3D model system of the developing placenta and its susceptibility to emerging pathogens.

### INTRODUCTION

The human placenta forms a barrier between mother and fetus that nourishes the embryo through the exchange of nutrients and gases and protects the fetus from harmful assaults. Placental abnormalities in the first trimester are associated with pregnancy complications such as preeclampsia, miscarriage, and fetal growth restriction. However, the ability to study human placental development in utero is limited due to ethical and legal concerns, particularly at the early stages. In recent years, two complementary approaches have been developed to model human placental development in vitro. The first approach is to derive 3D placental organoids from primary proliferative cells of the first-trimester placenta, which are called cytotrophoblasts (CTB). These trophoblast organoids contain CTBs that differentiate into syncytiotrophoblasts (STBs), much like the primary villi, and secrete placental hormones. The recent derivation of human trophoblast stem cells (hTSCs) offers an alternative approach to modeling the first-trimester placenta in vitro. hTSCs are isolated from primary placentas of the first trimester or human blastocyst outgrowths and can be maintained by combining a WNT activator, epidermal growth factor (EGF), and inhibitors of histone deacetylases (HDACs) and transforming growth factor beta (TGF-β). We and others have demonstrated that hTSCs can also be derived from naïve human pluripotent stem cells (hPSCs) or somatic cells via direct reprogramming. Here we explored whether hTSCs have the potential to self-organize into self-renewing 3D trophoblast organoids and determined the cellular complexity of the resulting organoids by single-cell transcriptomics. The findings indicate that, regardless of their source, hTSCs robustly generate 3D trophoblast organoids with a similar villous architecture similar to primary trophoblast organoids. These stem cell-derived trophoblast organoids (SC-TOs) comprise five distinct subpopulations that correspond to trophoblast cell types found during early pregnancy. X chromosome inactivation (XCI) dynamics during the generation of SC-TOs from naïve hPSCs are also characterized and approaches for their directed differentiation into invasive 3D EVT organoids are described. The generation of trophoblast organoids from naïve hPSCs provides an accessible and patient-specific 3D model system of human placental development.

In addition to mediating maternal-fetal communication, the placenta serves as an important defense against fetal viral infections. Transplacental infection of the Zika virus (ZIKV) has been implicated as one of several possible mechanisms underlying ZIKV vertical transmission during 4 the first trimester, which can result in severe congenital malformations such as microcephaly in infants. Coronavirus disease 2019 (COVID-19) is a severe acute respiratory disease caused by the SARS-CoV-2 virus. Since the first reported case in Wuhan, China, in December 2019, COVID-19 has rapidly become a global pandemic. While primarily associated with respiratory failure, recent studies have reported an alarming frequency of pregnancy complications in women who contracted COVID-19. SARS-CoV-2 utilizes ACE2 and TMPRSS2 surface proteins as entry factors and a subset of placental cells express these two proteins on their surface. Viral spike RNA and protein in ACE2-expressing maternal and fetal cells in placentas from SARS-CoV-2-infected women were recently detected, which correlated with alterations of the local renin-angiotensin system. However, clinical evidence of vertical transmission from mother to fetus appears limited in most circumstances, although it has been documented in some cases, particularly when there are comorbidities. These data demonstrate the need for an accessible in vitro model system to test mechanisms of SARS-CoV-2 infection of the placenta. Stem cell-derived organoid models have been used to test cell type-specific vulnerability to SARS-CoV-2 infection in other organ systems, such as the lung, gut, brain, and airway epithelium. Here, it is demonstrated that early trophoblast cell types in SC-TOs are readily infected by ZIKV but display limited susceptibility to SARS-CoV-2. These findings suggest that the first-trimester placenta may exhibit differential susceptibility to emerging pathogens and our model provides a foundation for elucidating the pathogenesis of viral infection.

Results

3D Trophoblast Organoids Derived from Naïve and Primary hTSCs Display Comparable Tissue Architecture, Placental Hormone Secretion, and Capacity for Long-Term Self-Renewal

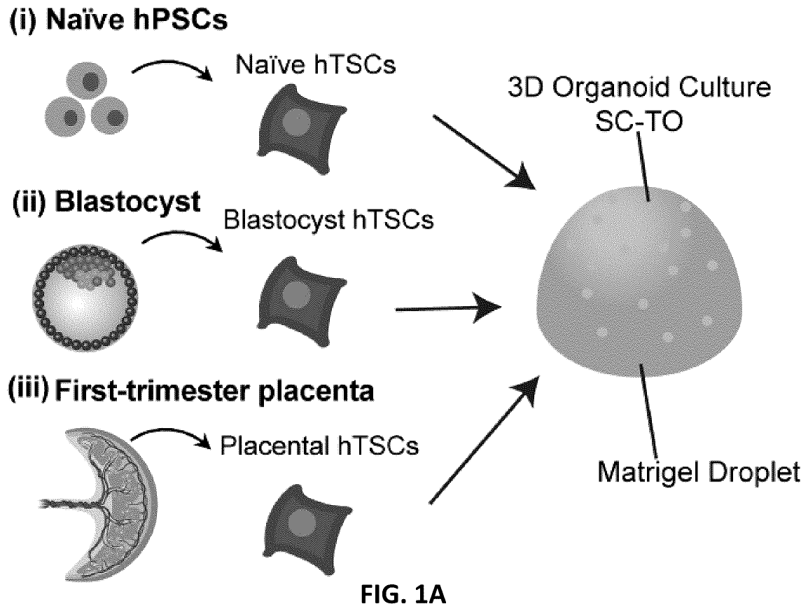

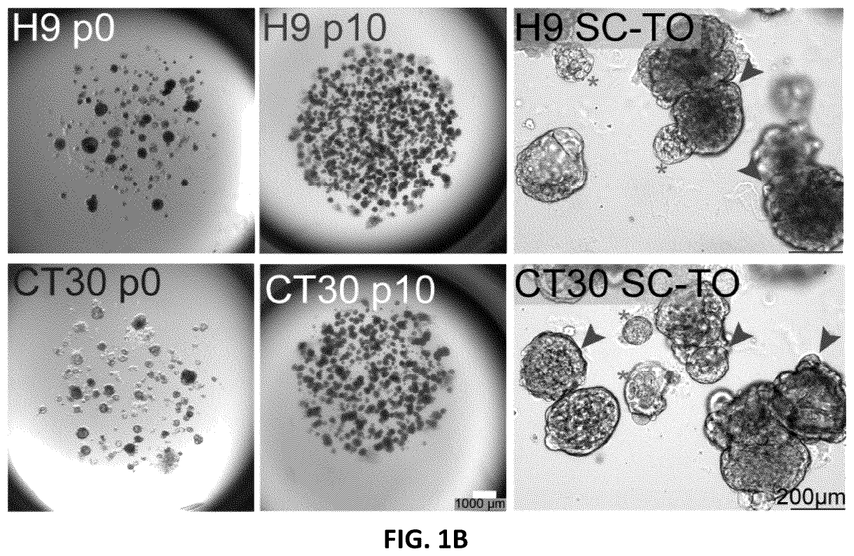

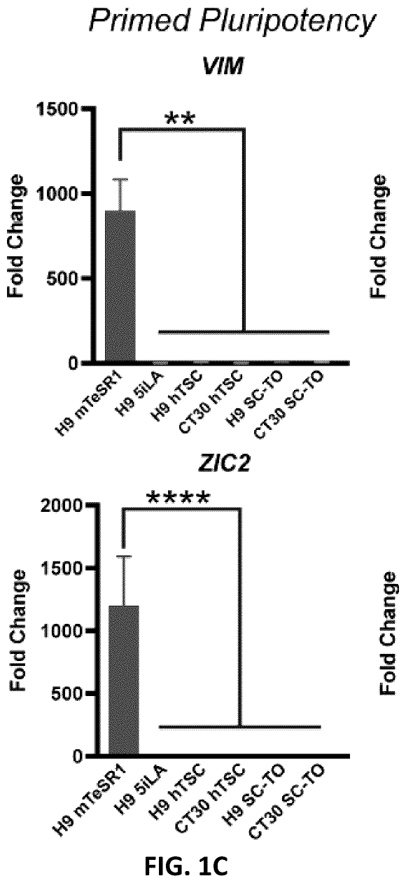

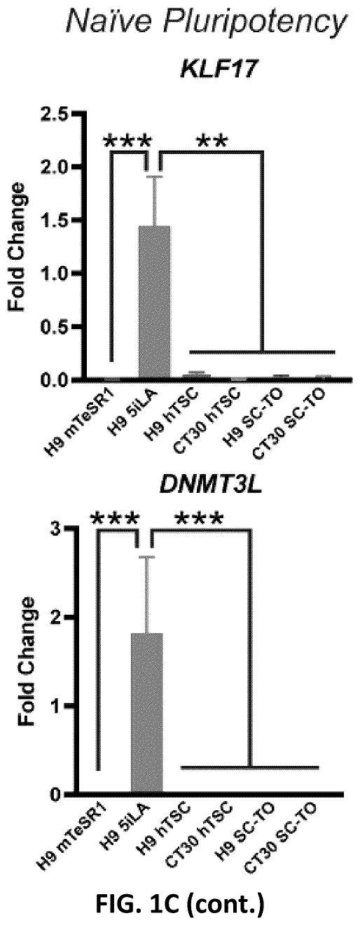

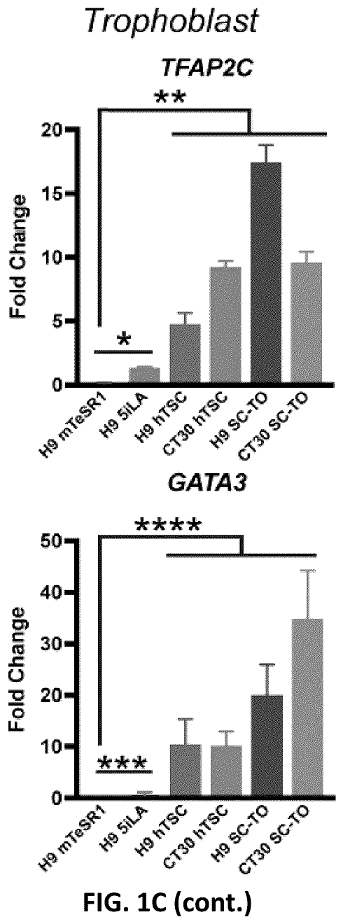

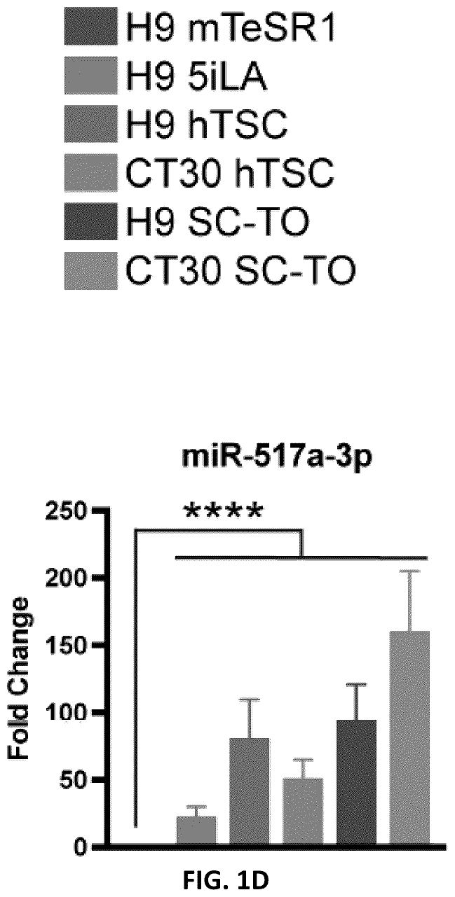

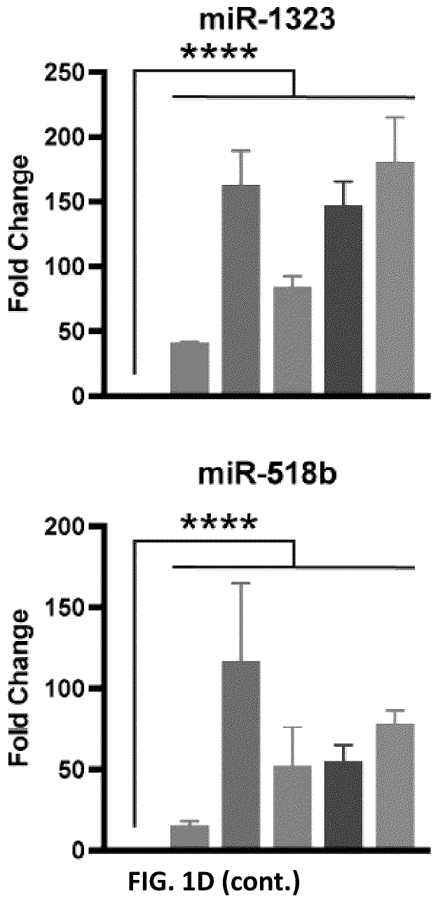

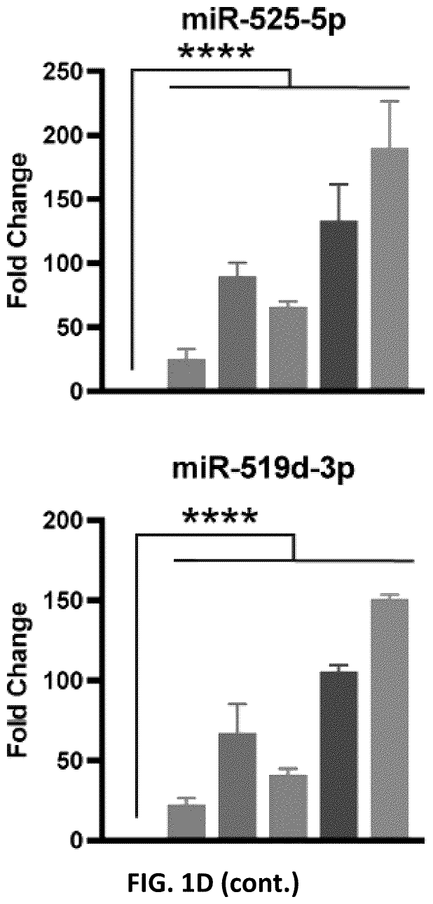

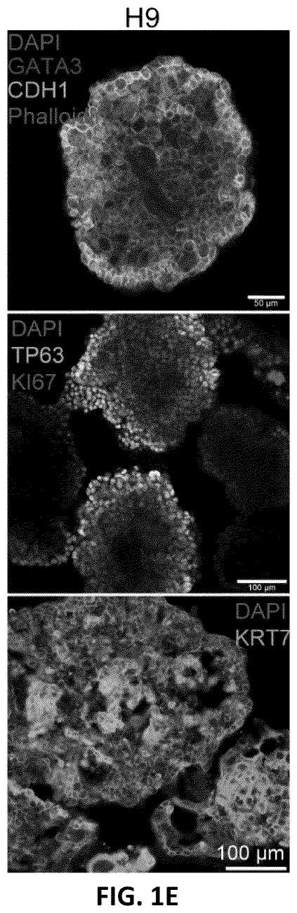

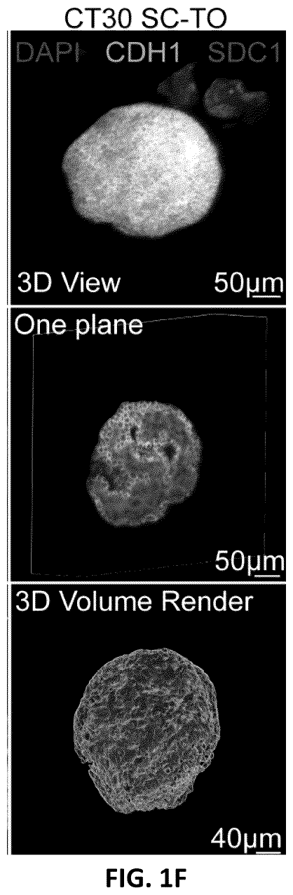

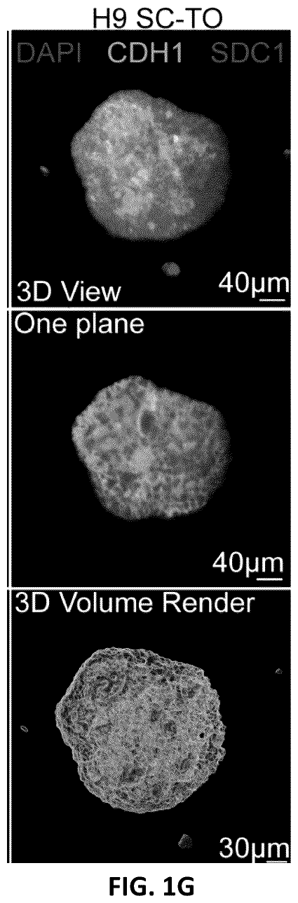

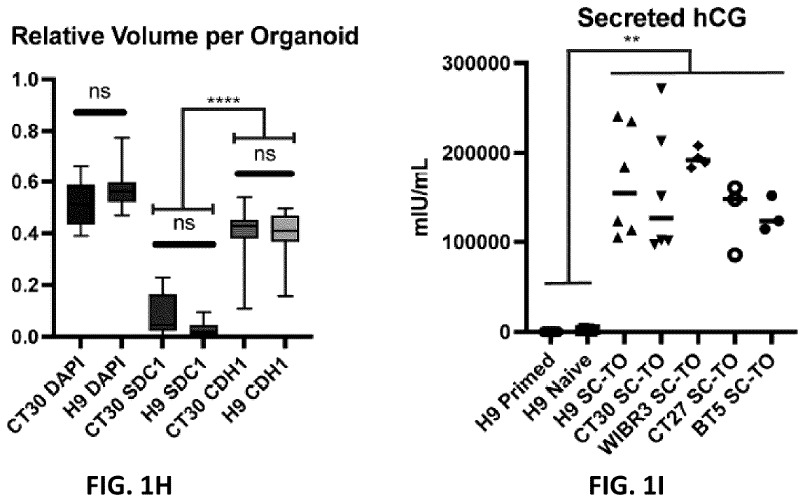

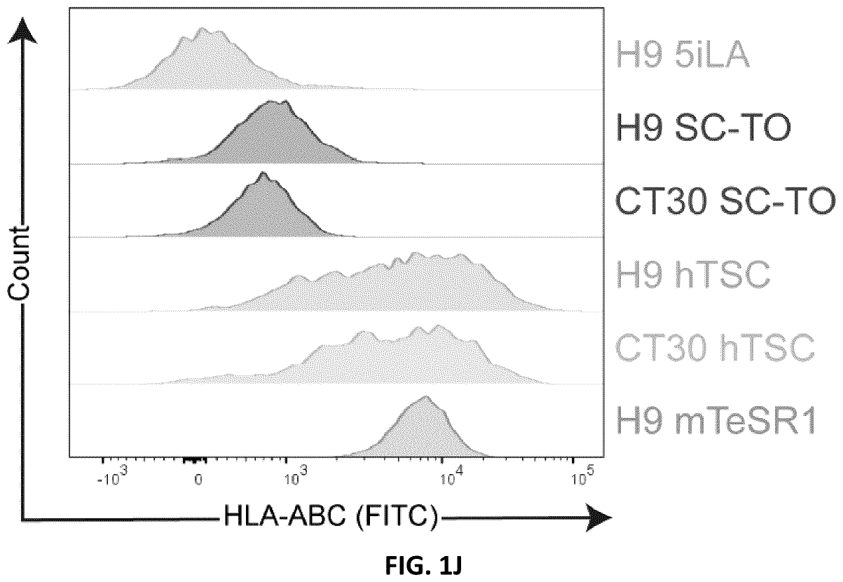

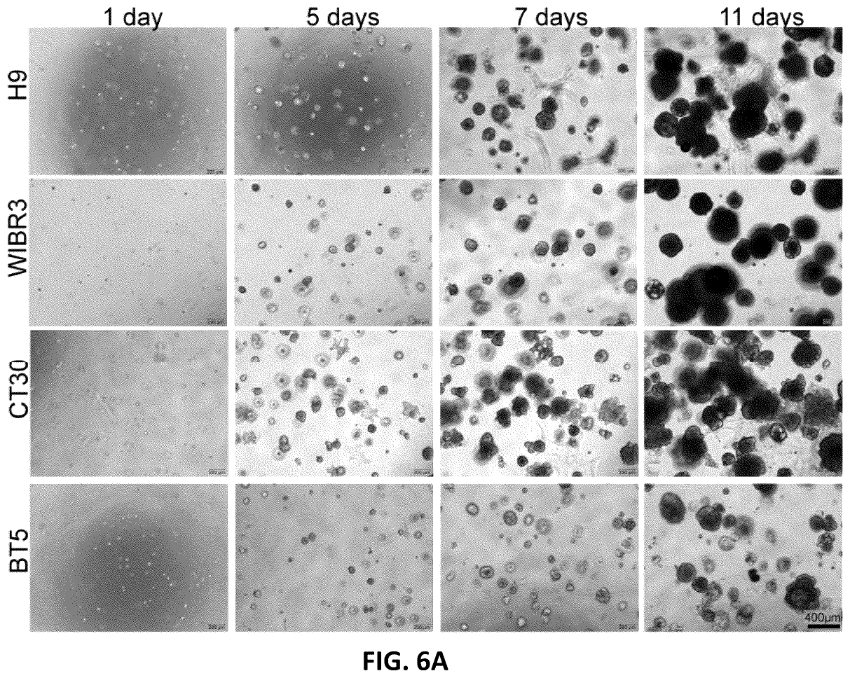

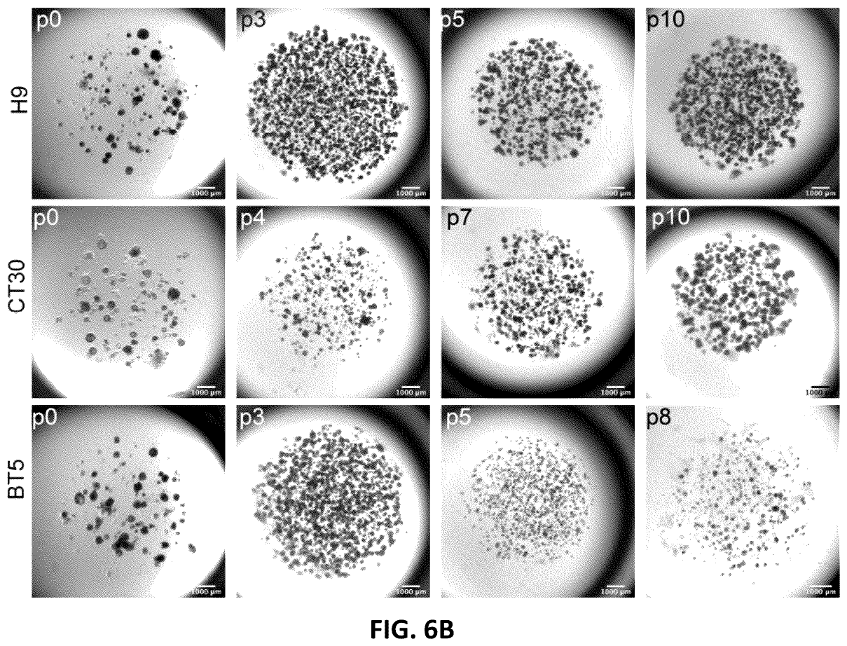

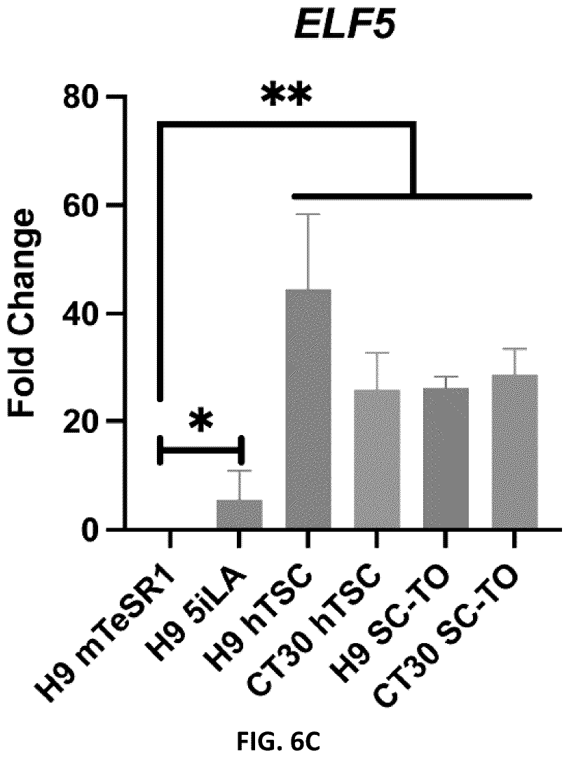

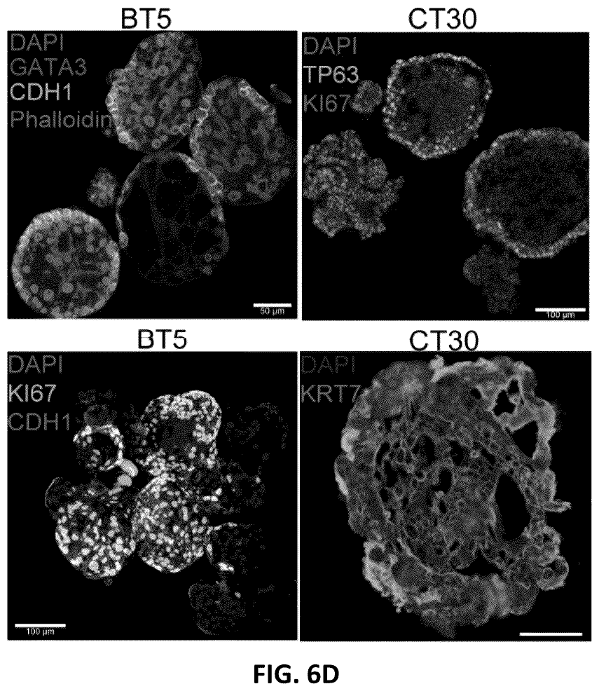

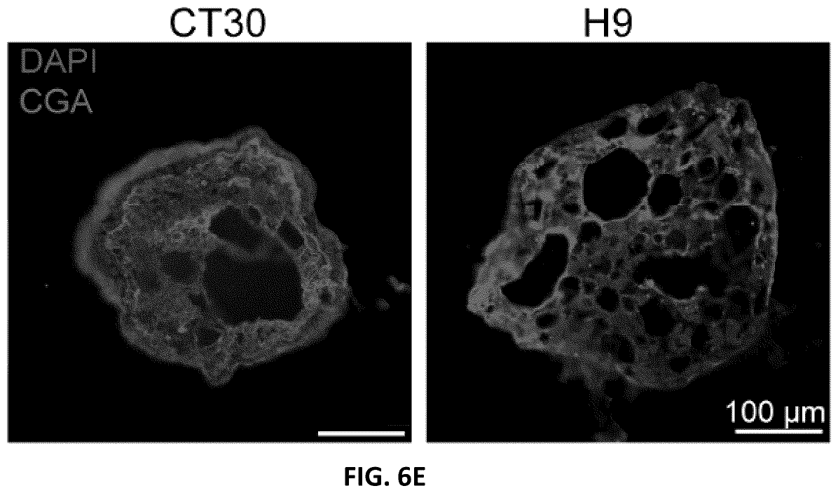

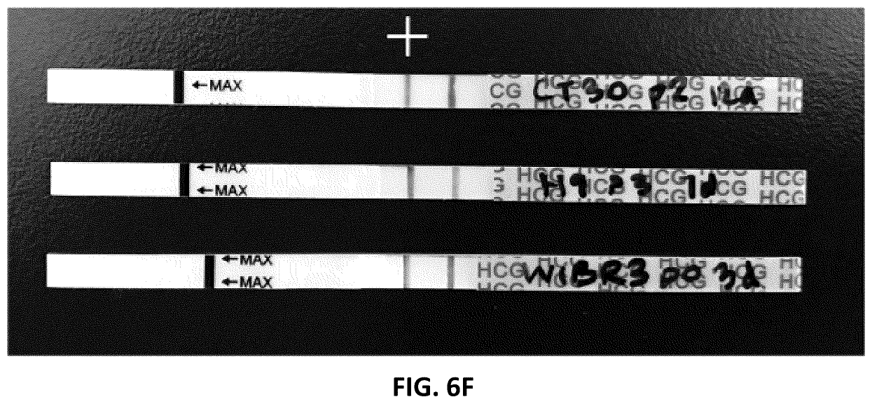

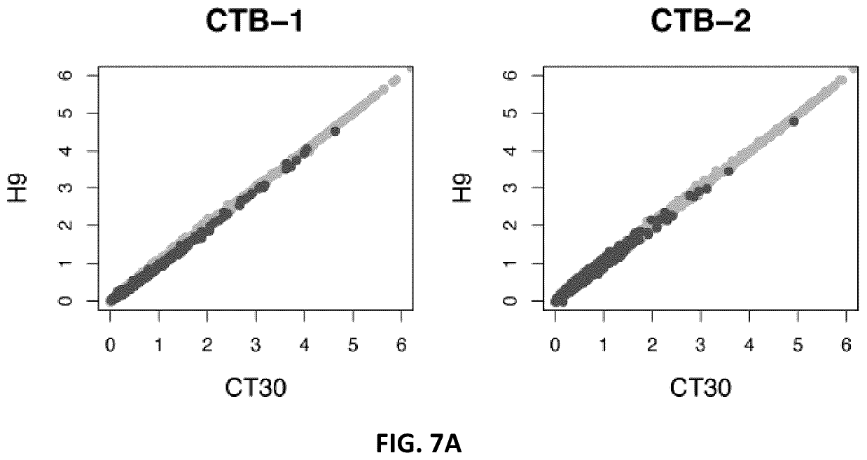

To explore the potential of hTSCs to self-organize into 3D organoids, hTSCs derived from a blastocyst (BT5), primary first-trimester CTBs (CT27, CT30), and naïve hPSCs (H9, WIBR3) were dissociated into single cells and seeded in 3D Matrigel droplets in the presence of trophoblast organoid medium (TOM) (FIG. 1A). 3D structures developed over the course of 10-12 days that could be maintained for at least 10 passages (FIGS. 1B and 6A-B). While most organoids had a dense morphology (FIG. 1B, red arrows), a subset formed cyst-like structures (FIG. 1B, asterisks). Quantitative real-time PCR (qRT-PCR) analysis confirmed the upregulation of trophoblast markers ELF5, GATA3, and TFAP2C and the downregulation of naïve and primed pluripotency markers in trophoblast organoids generated from naïve hPSCs (FIGS. 1C and 6C). In addition, all stem cell-derived trophoblast organoids (SC-TOs) showed substantial upregulation of a primate-specific microRNA (miRNA) cluster on chromosome 19 (C19MC), which exhibits placenta-specific expression (FIG. 1D). Interestingly, these miRNAs were already significantly induced in naïve compared to primed hPSCs, which may contribute to the trophoblast potential of naïve hPSCs. Immunofluorescence (IF) analysis indicated that epithelial CTBs (marked by CDH1 and TP63) were largely confined to the periphery of the organoids, while multinucleated STBs were located towards the interior (FIG. 1E). Furthermore, Ki67 staining demonstrated that this epithelial CTB compartment contained proliferative cells (FIGS. 1E and 6D). This “inside-out” architecture is reminiscent of the villous architecture described in primary trophoblast organoids derived directly from first-trimester placental tissues. Pan-trophoblast markers GATA3 and KRT7 were strongly expressed among the nuclear and cytoskeletal compartments, respectively (FIGS. 1E and 6D). Cyst-like structures present within the organoid culture were uniformly positive for the STB marker CGA, which encodes the alpha subunit of hCG (FIG. 6E) and thus represent a subset of fully syncytialized SC-TOs.

To obtain a more quantitative measurement of the distribution of key lineage markers, light-sheet imaging was performed on optically cleared SC-TOs. This analysis confirmed their predominant inside-out morphology with an outer shell of CDH1-positive cells encircling an inner syncytial compartment marked by SDC1 (FIG. 1F-G). Quantification of the relative volumes occupied by each marker revealed a 5-10 fold increase in CDH1 compared to SDC1 per organoid, but no significant differences were observed between SC-TOs obtained from naïve and primary hTSCs (FIG. 1H). The secretion of human chorionic gonadotropin (hCG), a key placental hormone, was confirmed using an over-the-counter pregnancy test and by hCG ELISA (FIGS. 1I and 6F). Finally, flow cytometry analysis for classical HLA class I surface antigens was performed, which are weakly expressed in human trophoblast cells but more significantly in amniotic epithelial cells. While significant HLA-ABC expression was detected in naïve and primary hTSCs, SC-TOs displayed a reduction in mean signal intensity (FIG. 1J). This suggests that HLA-ABC expression is stimulated by 2D hTSC culture and that SC-TOs more faithfully recapitulate the HLA expression profile of human trophoblast cells in vivo. Taken together, these data demonstrate that all examined hTSC lines harbor the potential for self-organization into 3D SC-TOs with comparable gross morphological structure, placental hormone secretion, and capacity for long-term self-renewal.

Single-Cell Transcriptomes Reveal the Cellular Complexity of 3D Organoids Generated from Naïve and Primary hTSCs

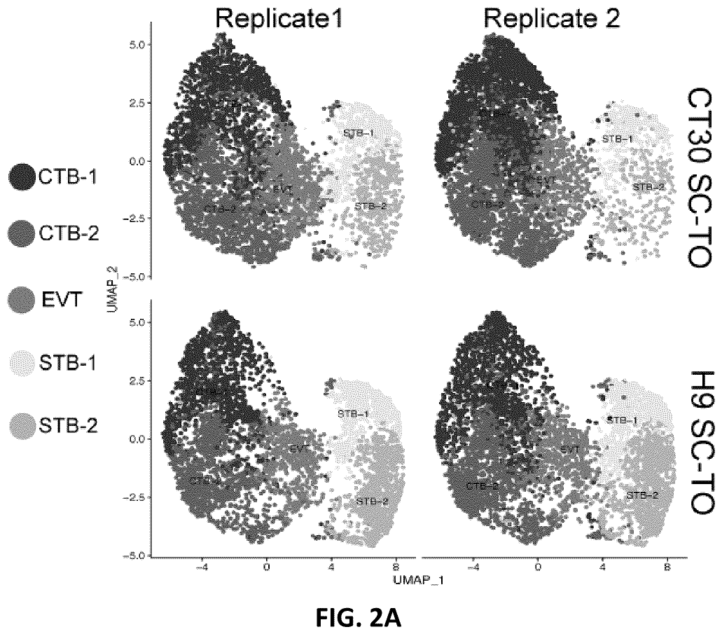

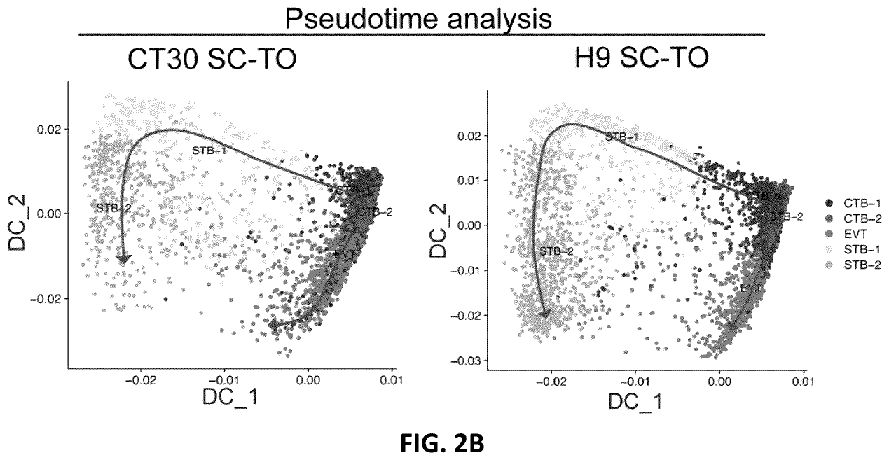

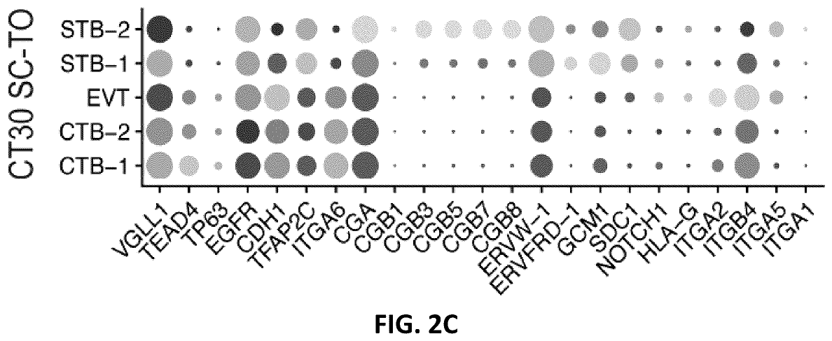

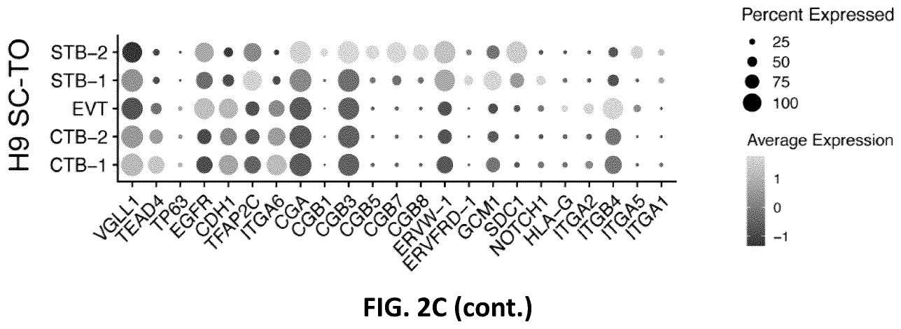

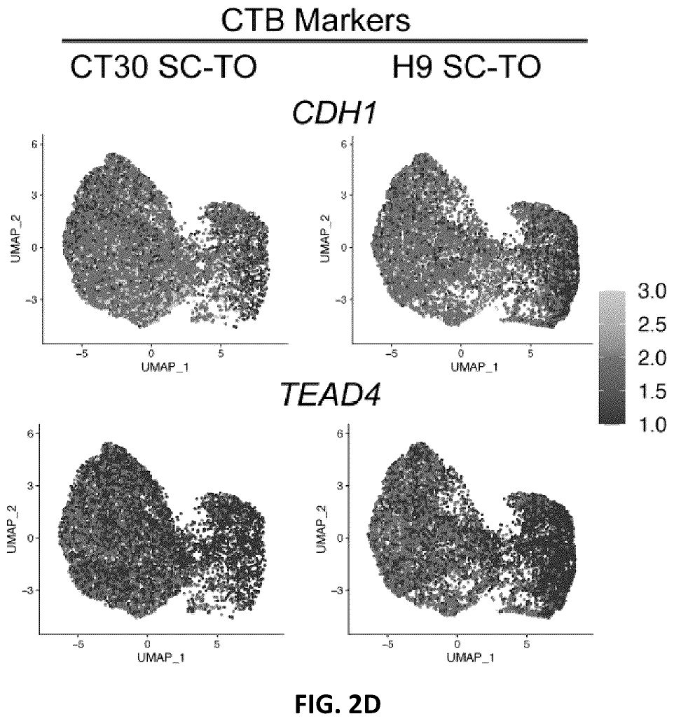

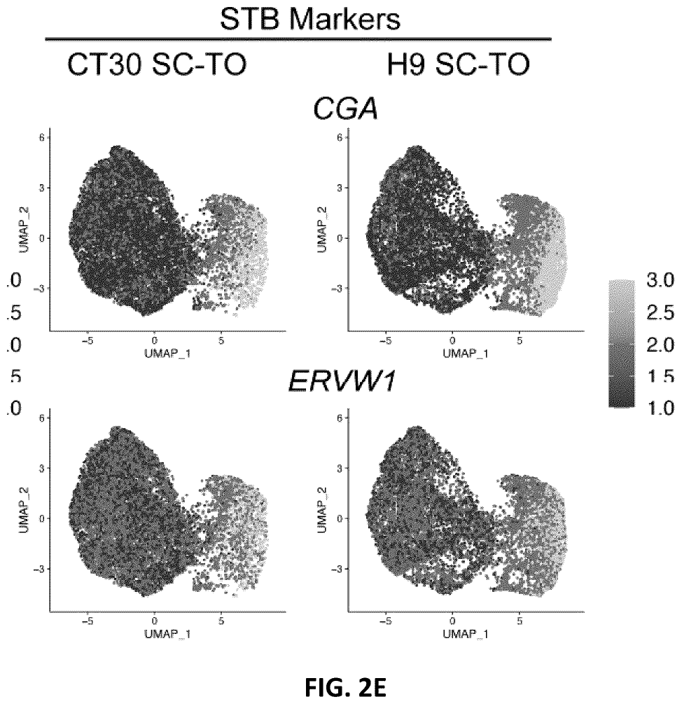

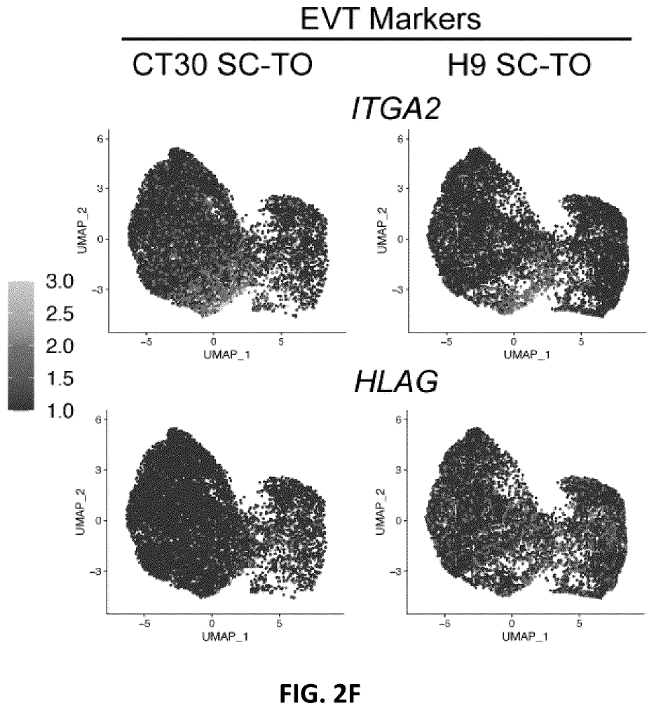

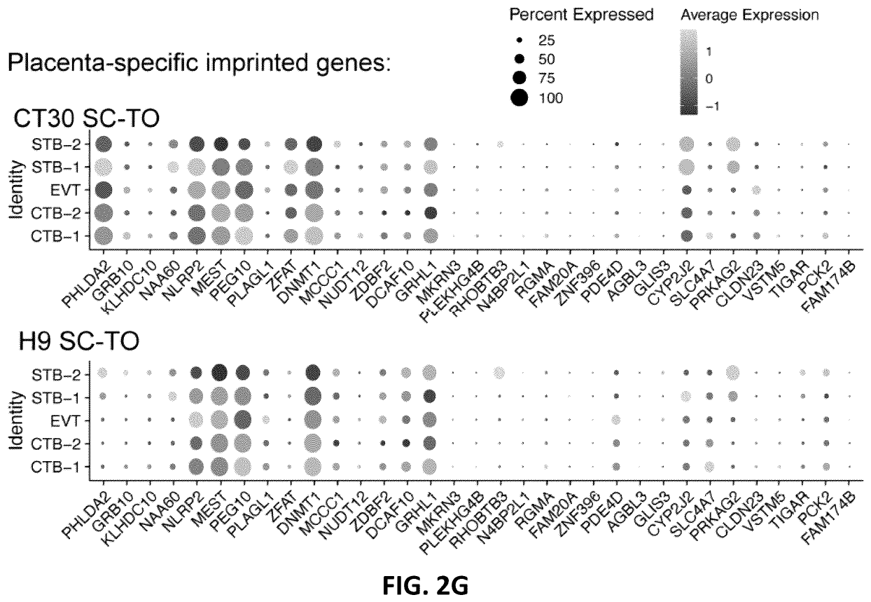

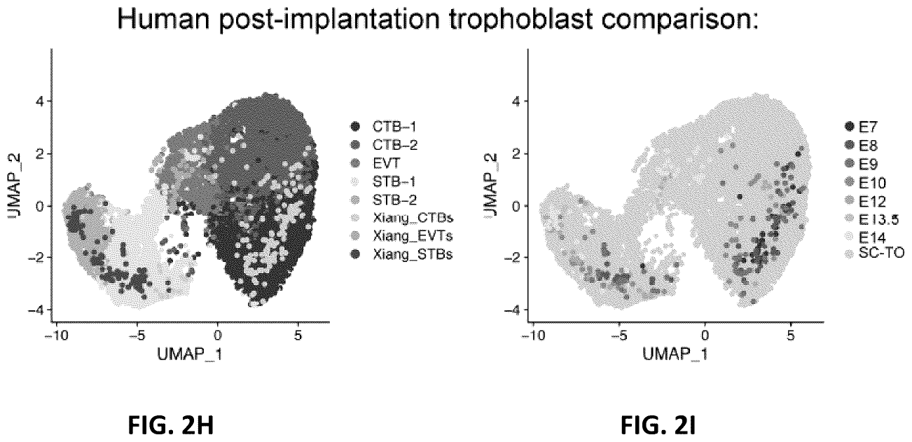

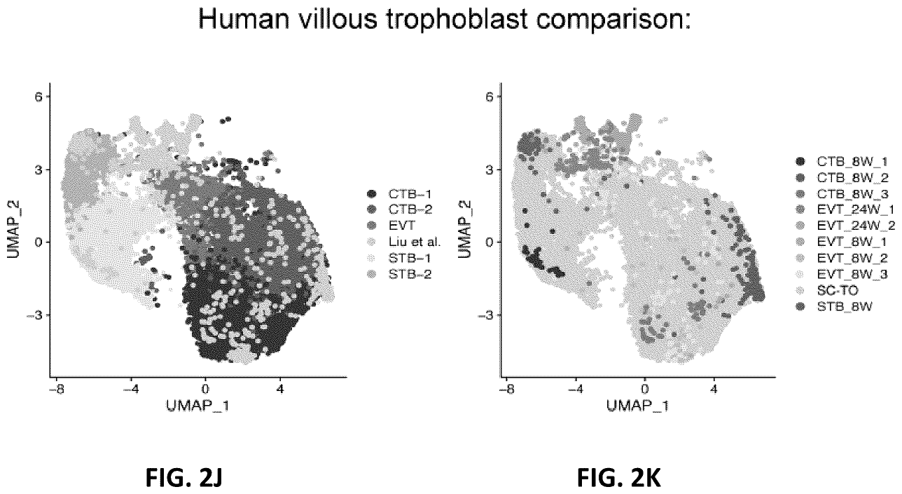

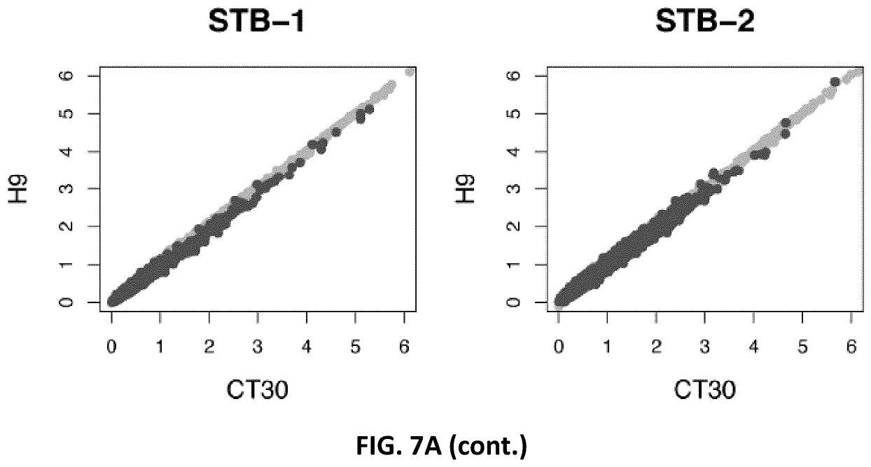

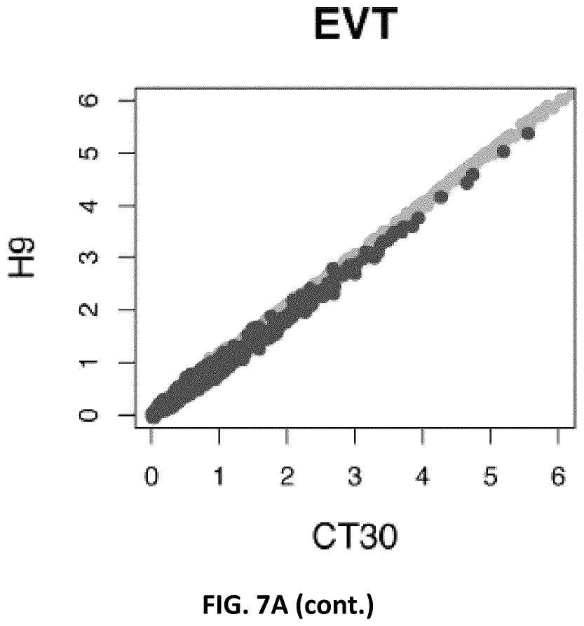

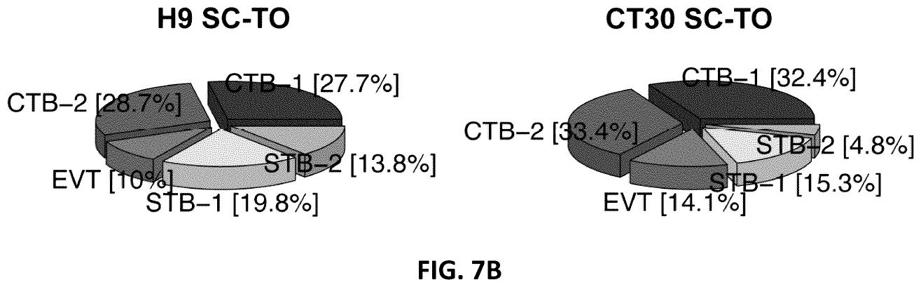

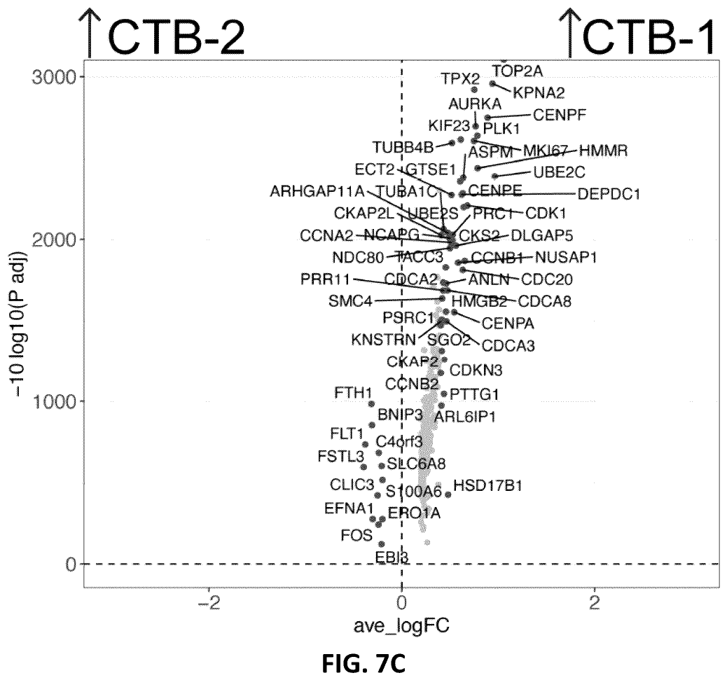

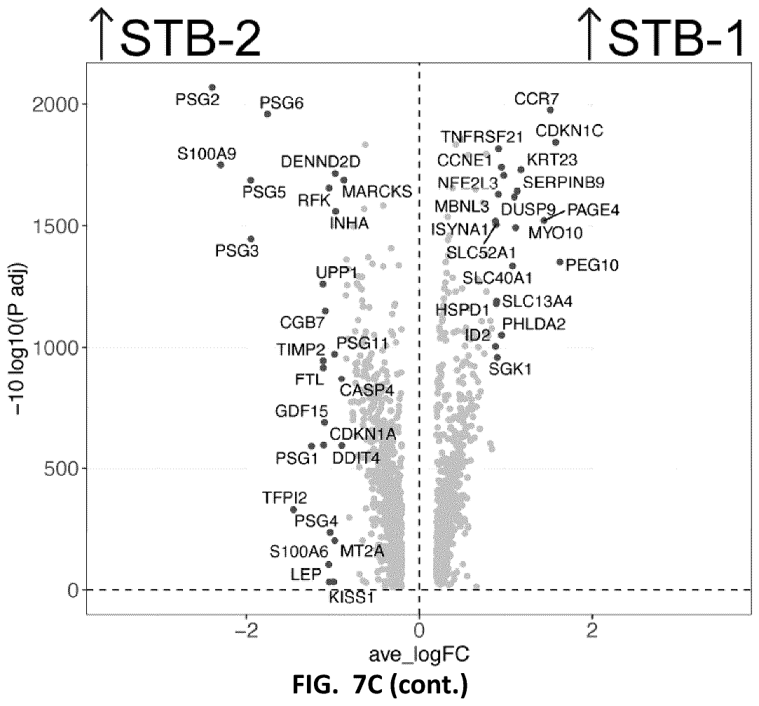

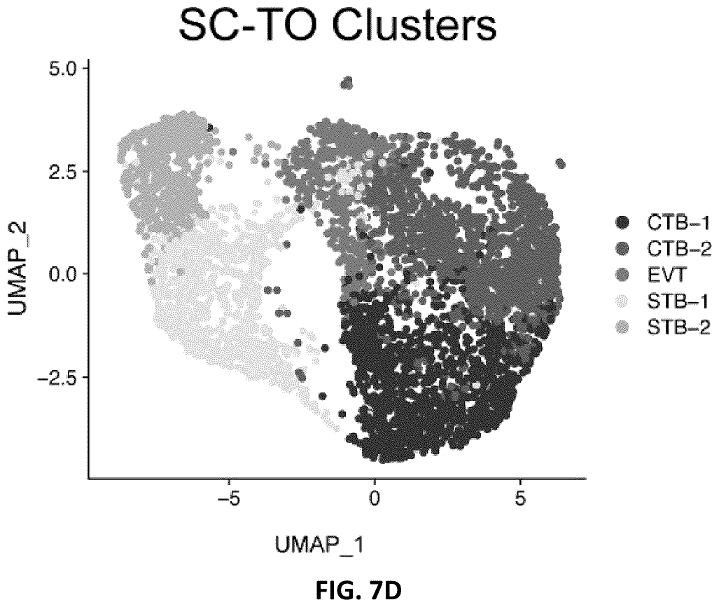

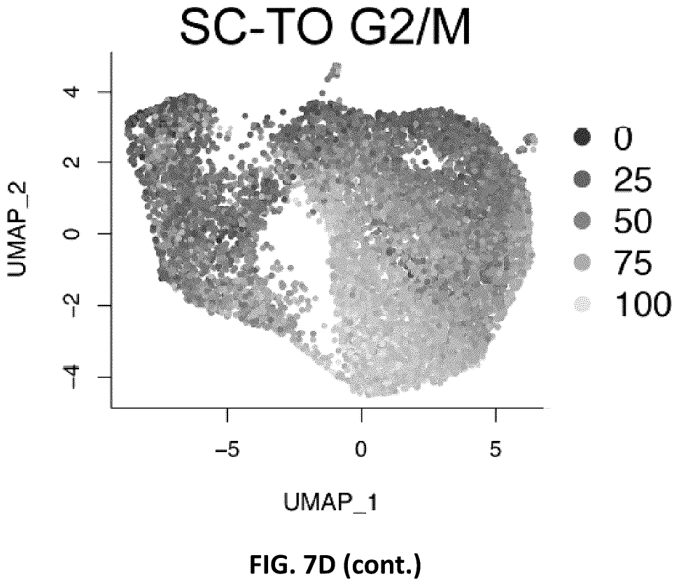

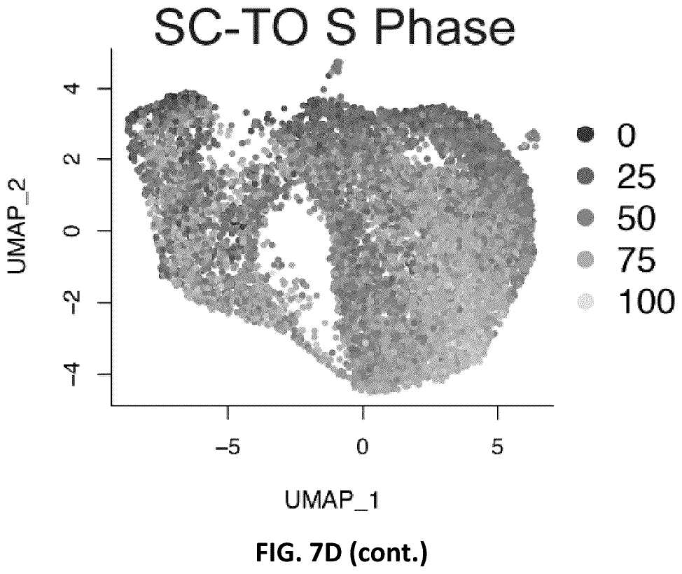

The cellular composition of SC-TOs generated from naïve and primary hTSCs was examined by single-cell transcriptome analysis. Organoids obtained from naïve hTSCs (H9) and primary hTSCs (CT30) were dissociated into single cells and cDNA libraries were generated for single-cell RNA-sequencing (scRNA-seq) using the 10× Genomics platform. Dimensional reduction analysis by Uniform Manifold Approximation and Projection (UMAP) revealed a very similar cellular composition in SC-TOs generated from naïve and primary hTSCs, which was highly reproducible between two replicates (FIG. 2A). Both H9 and CT30 SC-TOs comprised five discrete trophoblast subpopulations: two CTB clusters, two STB clusters, and a small EVT cluster. Pseudotime analysis revealed two distinct developmental trajectories, both of which emerged from CTB-1 (FIG. 2B). One of these trajectories passes from CTB-1 directly into STB-1 and subsequently into STB-2, while the other trajectory proceeds via CTB-2 into EVT. This analysis suggests that trophoblast progenitors within CTB-1 bifurcate into either an EVT or STB trajectory. The CTB clusters were marked by elevated expression of genes such as CDH1, ITGA6, TEAD4, and VGLL1 (FIG. 2C-D); the STB clusters were marked by high expression of CGA, CGB1-8, GCM1, ERVW-1, and SDC1 (FIGS. 2C and E); and the EVT cluster exhibited elevated expression of HLAG, ITGA2, and NOTCH1 (FIGS. 2C and F). However, the absence of mature EVT markers such as ITGA1 and the retention of epithelial surface markers CDH1 and ITGB4 in this cluster (FIGS. 2C and D) suggest that these cells have not yet completed differentiation into fully invasive EVTs. This cluster is therefore referred to as primitive EVTs. Differential gene expression analysis between matched clusters in H9 and CT30 organoids revealed few significantly differentially expressed genes (DEGs) (FIG. 7A), although the overall proportion of STBs was slightly increased in H9 SC-TOs (FIG. 7B). Hence, SC-TOs generated from naïve and primary hTSCs display a similar cellular composition at the single cell level.

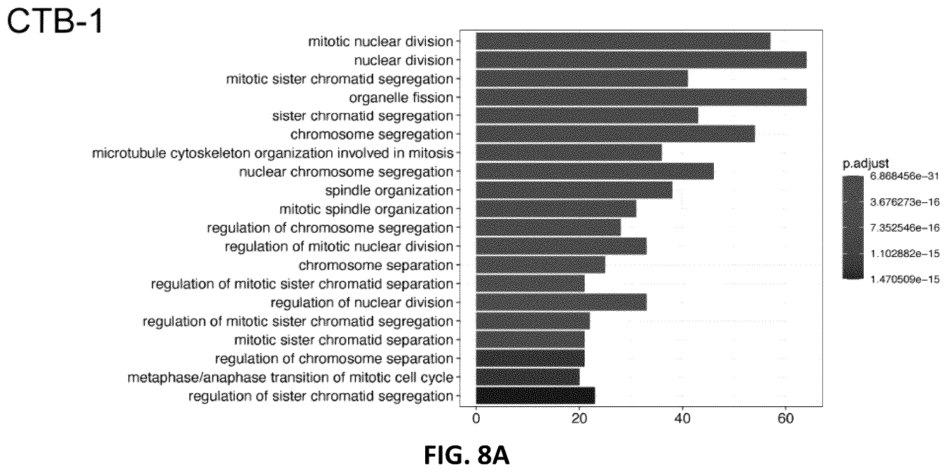

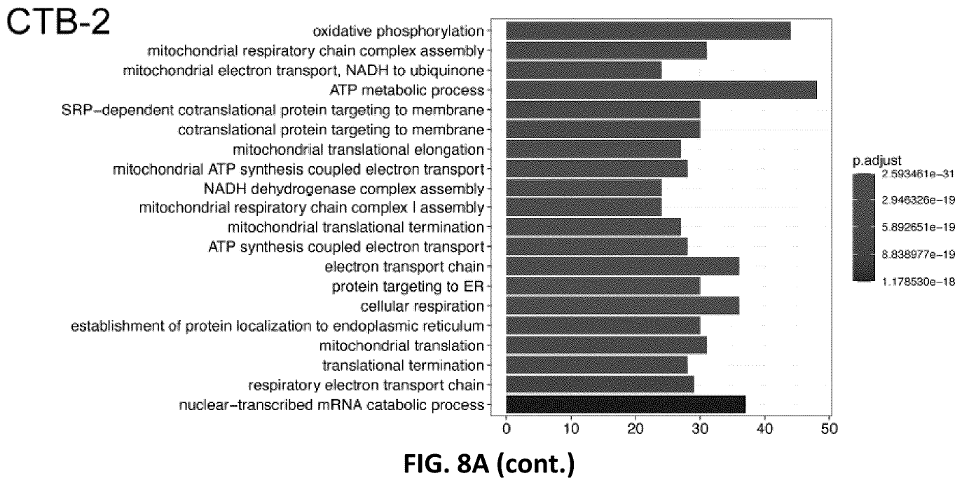

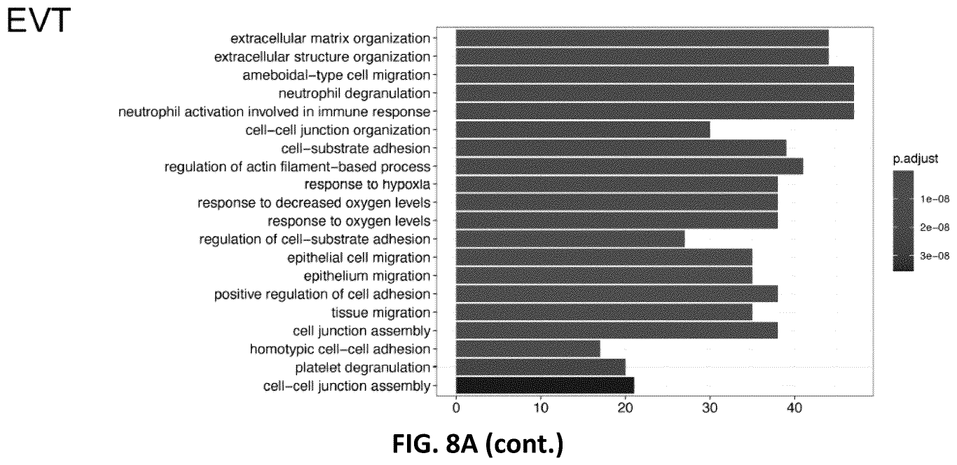

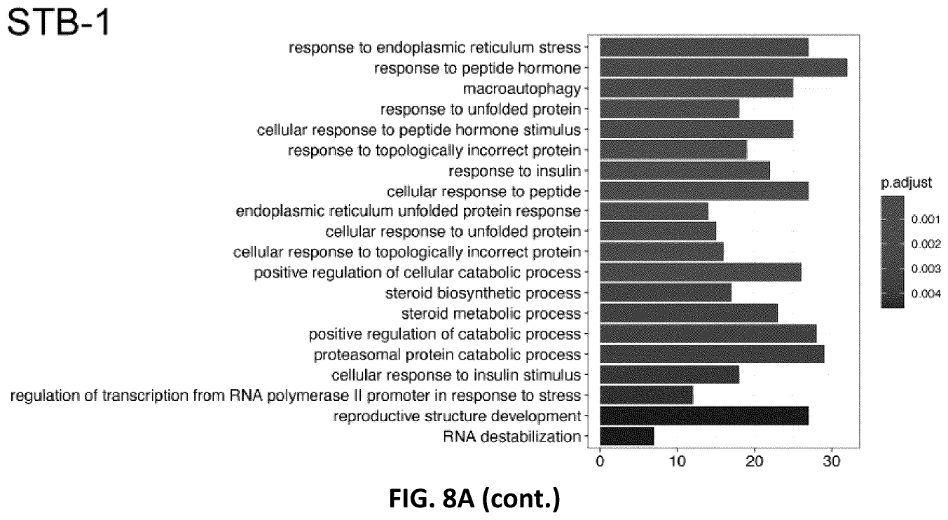

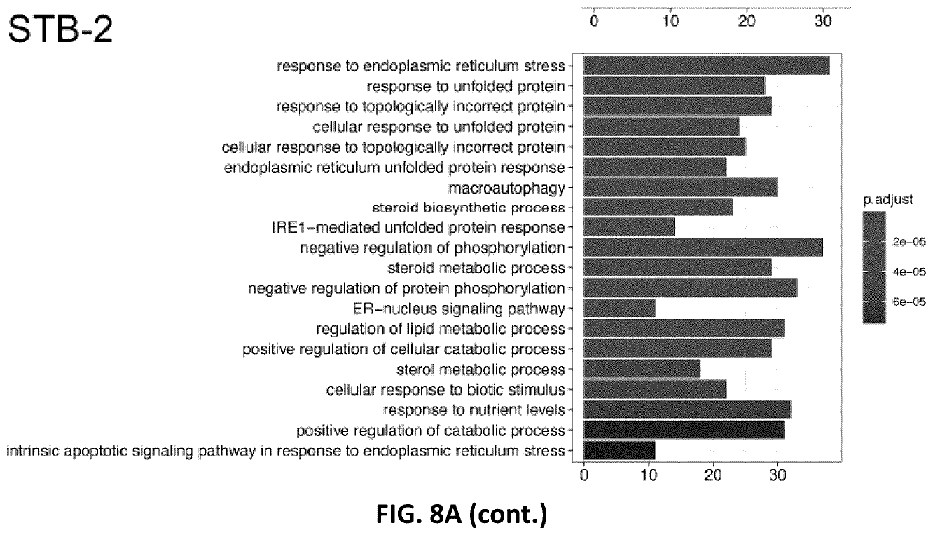

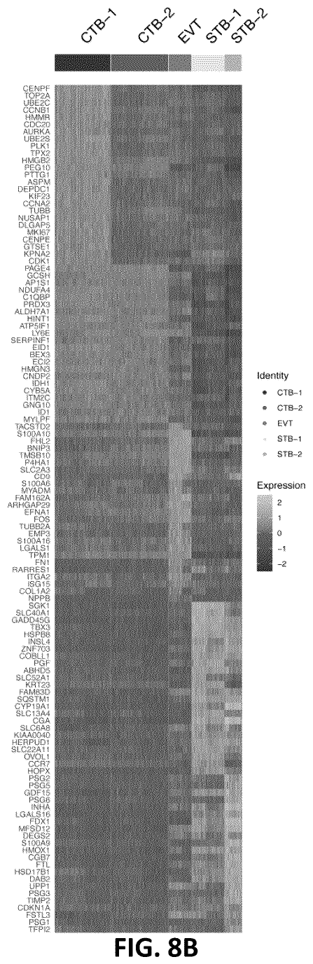

To further define the identity of these distinct trophoblast subpopulations, DEGs between the two CTB subpopulations (CTB-1 vs. CTB-2) were analyzed. Volcano plot analysis indicated that the CTB-1 cluster was enriched in cell cycle-related genes, such as MKI67, CDK1, CCNA2, and CDC20 (FIG. 7C). By analyzing genes associated with distinct stages of the cell cycle, we found that CTB-1 was specifically enriched in transcripts related to G2/M phases (FIG. 7D). Gene Ontology (GO) analysis based on DEGs further corroborated that the CTB-1 cluster was enriched in mitotic processes, while CTB-2 displayed a metabolic shift towards oxidative phosphorylation (FIG. 8A). These findings suggest that the CTB-1 cluster represents proliferative CTBs, while CTB-2 contains trophoblast progenitors that have exited the cell cycle and are transitioning towards a more specialized fate. In contrast, GO analysis on the primitive EVT cluster showed upregulation of pathways involved in ECM organization, migration, immune cell interaction, and oxygen sensing, all of which are central processes for EVT function (FIG. 8A). DEGs between the two STB subpopulations (STB-1 vs. STB-2) were also analyzed. STB-2 showed the strongest induction of pregnancy-specific glycoprotein (PSG) family genes, genes encoding the alpha and beta subunits of hCG (CGA, CGB3, CGB5, CGB7, and CGB8), and leptin (LEP) (FIG. 7C). Furthermore, all cells in this cluster had exited the cell cycle, while a fraction of STB-1 cells still resided in G2/M phases (FIG. 7D). STB-1 was enriched in the expression of GCM1, ERVFRD-1, and OVOL1, which have been implicated in regulating the fusion of CTB progenitors into STBs (FIG. 2C, 8B. It is surmised that STB-1 represents a transitional population of CTBs that are fusing into STBs, which was also suggested by the pseudotime analysis (FIG. 2B), while STB-2 represents more differentiated STBs.

Naïve hPSCs are known to undergo erasure of parent-specific imprinting marks as a consequence of global DNA demethylation. However, a recent study reported that hTSCs derived from naïve hPSCs displayed only modest differences in the expression of placenta-specific imprinted genes. To corroborate these findings in our organoid model, we compared the expression of imprinted genes at the single cell level in SC-TOs generated from naïve and primary hTSCs (FIG. 2G). Overall, the two SC-TO lines exhibited highly correlated expression of placenta-specific imprinted genes with the notable exceptions of PHLDA2 and ZFAT. Interestingly, failure to activate ZFAT during hTSC derivation from naïve hPSCs was also noted by Pastor and colleagues. It is concluded that placenta-specific imprinted genes are largely expressed at appropriate levels during the generation of SC-TOs from naïve hPSCs.

### SC-TO Subpopulations Correspond to Trophoblast Identities in the Human Post-Implantation Embryo

The transcriptional correspondence between SC-TOs and discrete stages of human trophoblast development was evaluated in vivo. The organoid clusters were well-aligned with CTB, EVT, and STB identities in 3D human embryos cultured through implantation stages (FIG. 2H). When segregated by developmental time points, STBs at embryonic days (E)7-12 were more closely aligned with STB-1, while STBs at E13.5-14 instead clustered with the mature STB-2 subpopulation (FIG. 2I). The primitive EVT cluster was aligned with EVTs at E13.5-14, while both CTB clusters correlated with CTBs at E7-12 (FIG. 2I). Whether SC-TOs may correspond transcriptionally to trophoblast identities at later stages of human placental development was also examined. Proliferative CTBs isolated at 8 weeks of human placental development (CTB_8W_3) were aligned with CTB-1, while post-mitotic CTBs (CTB_8W_2) overlapped with CTB-2 (FIG. 2J-K). Liu et al. also identified a subpopulation of fusion-competent CTBs in vivo that expressed some syncytial markers, which they called CTB_8W_1. This subpopulation corresponded to STB-1, which further reaffirms the transitional nature of this cluster. In contrast, mature STBs were manually dissected from the 8-week-old placenta clustered with STB-2 (FIG. 2J-K). Finally, EVTs from the 8-week-old placenta were broadly distributed across the EVT and CTB clusters. SC-TO EVTs overlapped marginally with EVT_8W_2 and 3, which comprise a mixture of proliferative and immune-responsive EVTs. In contrast, SC-TO EVTs did not overlap with 24-week EVTs that had completely invaded the maternal endometrium. It is concluded that SC-TOs encompass a variety of trophoblast identities that correspond transcriptionally to the first-trimester placenta. The strongest correlation, however, was observed with trophoblast subpopulations in early post-implantation embryos.

XCI Dynamics During Trophoblast Organoid Generation from Naïve hPSCs

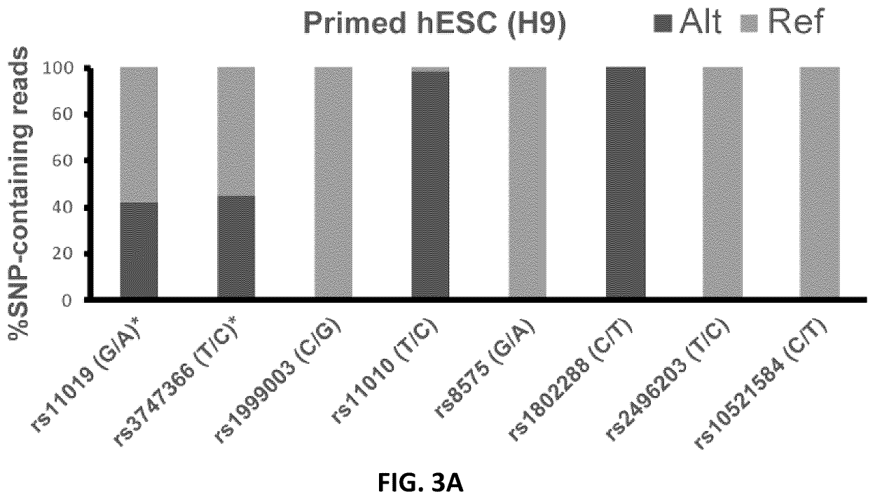

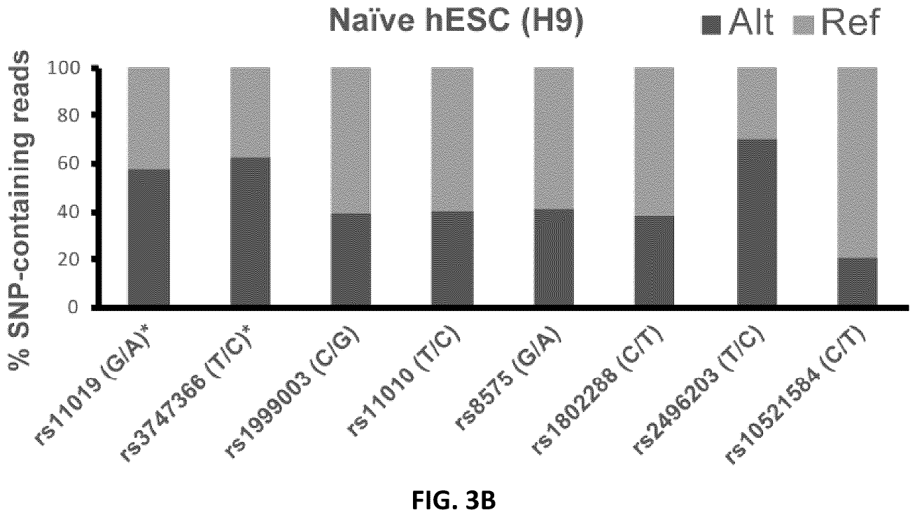

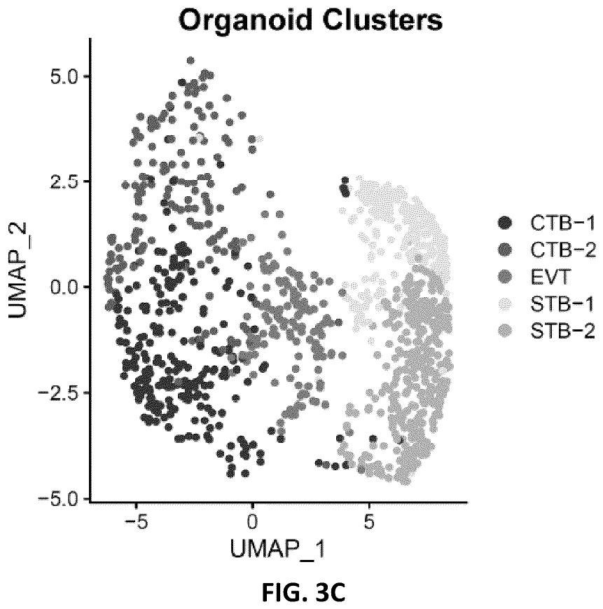

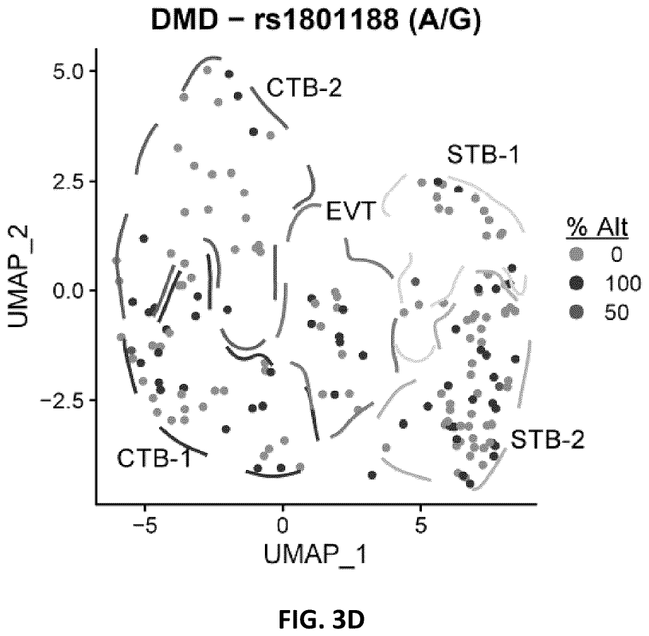

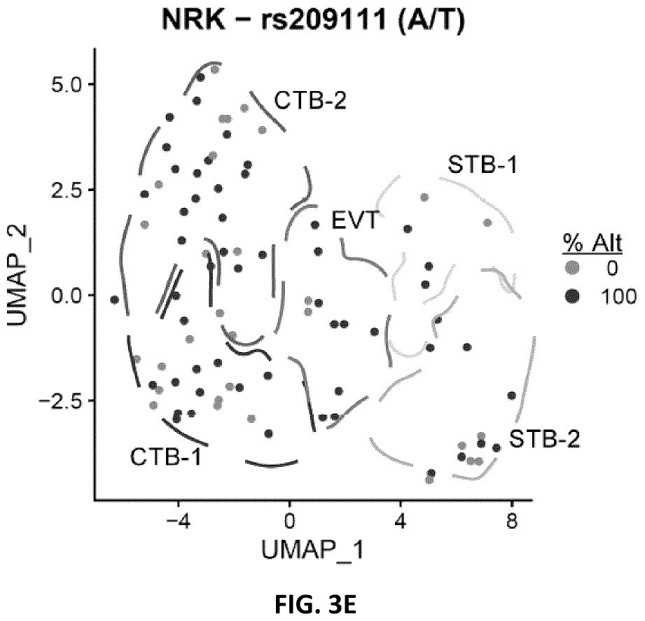

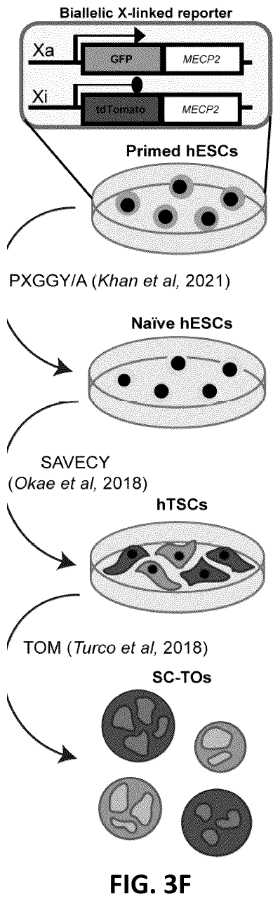

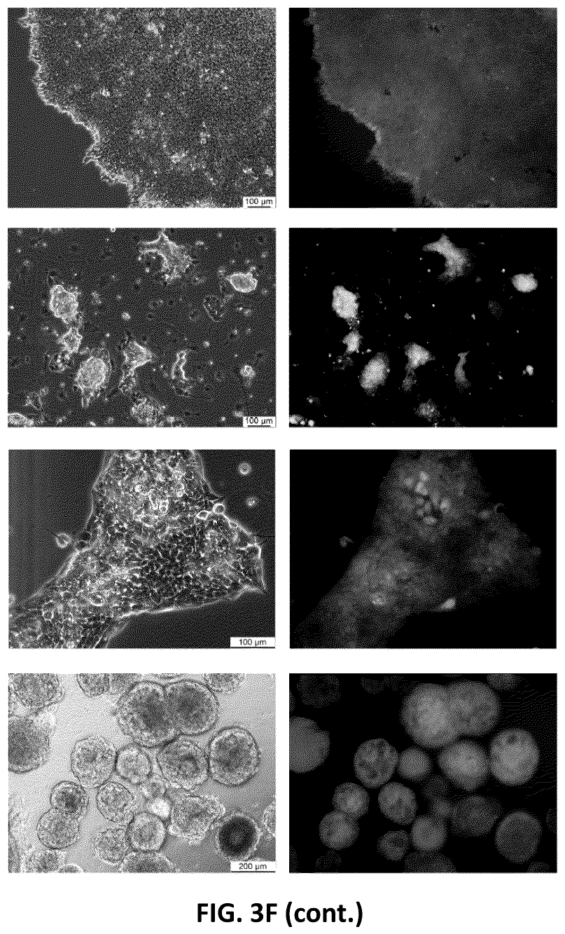

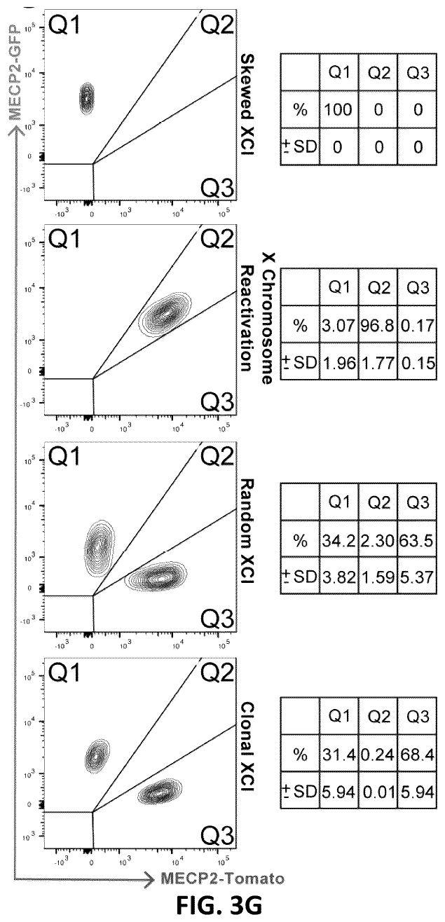

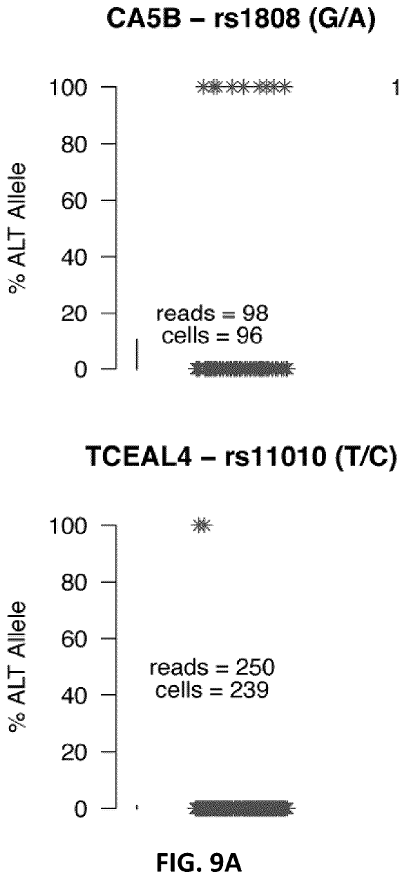

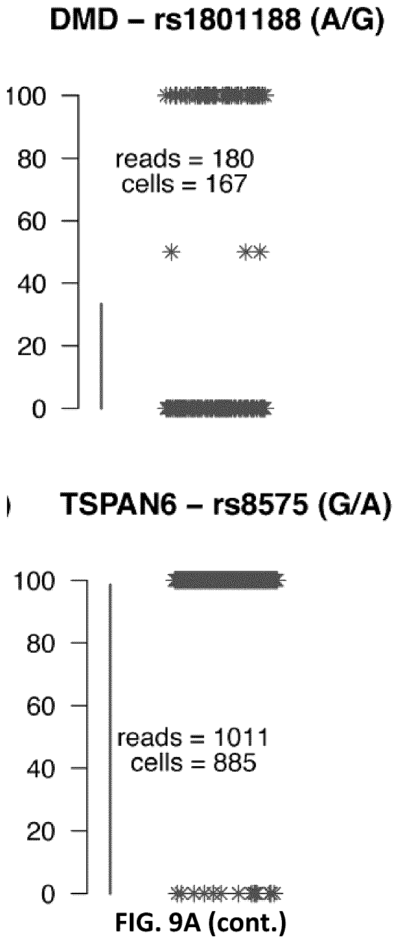

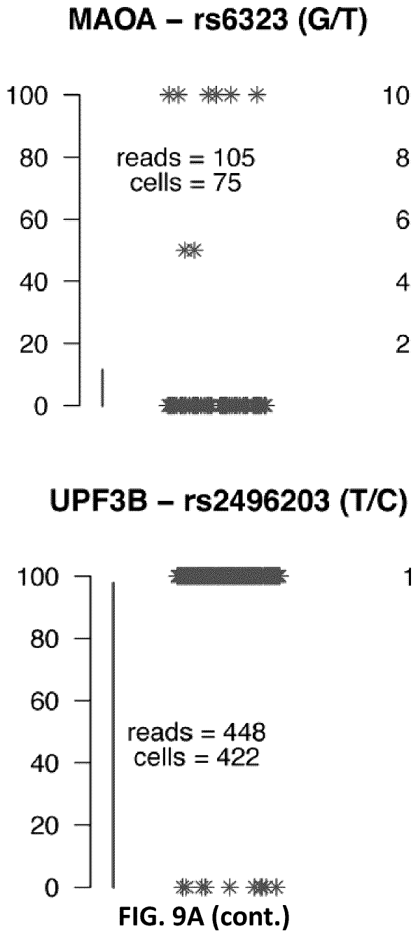

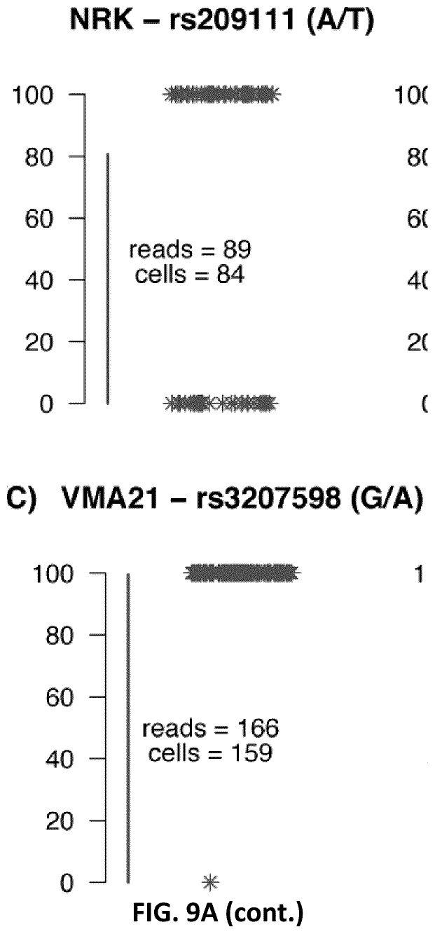

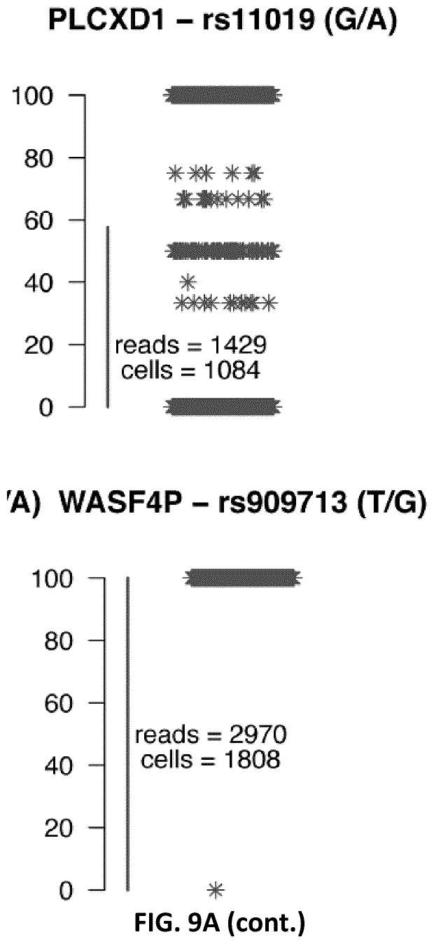

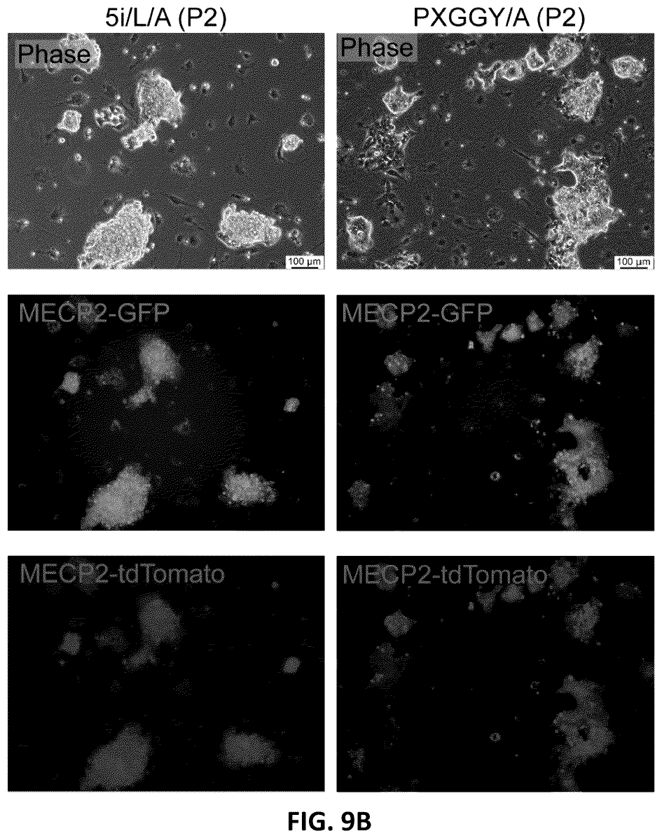

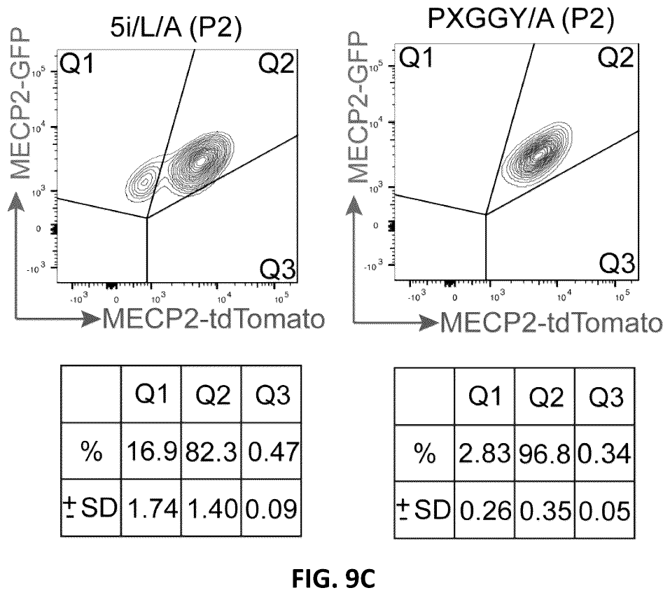

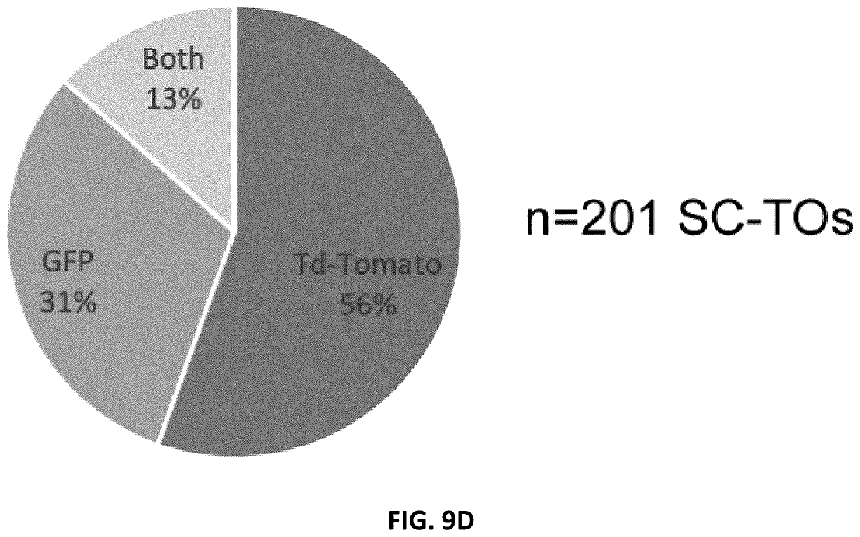

Like somatic tissues, female extraembryonic tissues must undergo XCI in order to achieve dosage compensation with males. In female mice, the paternal X chromosome is preferentially inactivated in the placenta and yolk sac. Several studies have reported that the human placenta at term is composed of relatively large clonal populations that express either the maternal or paternal X chromosome. However, XCI during human trophoblast development has not been studied thus far. Since naïve hPSCs are known to undergo X chromosome reactivation (XCR), it was postulated that it should be possible to model placental XCI dynamics during the generation of SC-TOs from naïve hPSCs. Taking advantage of a previously published single nucleotide polymorphism (SNP) array in H9 hESCs, we first confirmed using bulk RNA-sequencing data that induction of naïve pluripotency in 5i/L/A conditions resulted in a switch from mono-allelic to bi-allelic expression of X-linked genes (FIG. 3A-B). The XCI status of SC-TOs generated from these H9 naïve hESCs was then examined using our 10× single-cell transcriptome data. With the exception of PLCXD1, which is known to escape XCI, none of the examined X-linked transcripts showed substantial bi-allelic expression in SC-TOs (FIG. 9A). This confirms that XCI indeed takes place during trophoblast differentiation of naïve hESCs. Some of the examined transcripts showed a random XCI pattern with a heterogeneous expression of the reference and alternative alleles in all SC-TO subpopulations, including CTB-1 (FIG. 3C-E). This suggests that allele-specific expression of X-linked genes is already established within CTB progenitors and subsequently maintained upon EVT or STB differentiation. In contrast, other transcripts showed a more skewed XCI pattern with a predominant expression of a single allele (FIG. 9A).

To track XCI dynamics during SC-TO derivation from naïve hESCs, a bi-allelic reporter hESC line was used in which both alleles of the X-linked MECP2 gene are labeled with different fluorophores. This line is GFP-positive in primed cells and becomes double positive for GFP and tdTomato upon naïve reversion, indicating XCR. It was noticed that a subpopulation of non-reactivated cells remained present upon naïve reversion in 5i/L/A (FIG. 9B-C). However, the fraction of double-positive cells was enhanced using a recently developed alternative naïve induction cocktail, PXGGY/A (FIG. 9B-C). hTSCs derived from this homogeneous population of WIBR3 MECP2GFP/tdTomato naïve hESCs displayed an almost complete loss of bi-allelic expression and the emergence of discrete GFP-positive and tdTomato-positive cells (FIG. 3F-G). Hence, XCI at the MECP2 locus occurs at an early stage during trophoblast lineage induction in naïve hESCs in vitro, in agreement with recent observations in non-human primate embryos. Individual organoids derived from this mixed population of MECP2GFP and MECP2tdTomato hTSCs exhibited exclusive expression of either fluorophore, although a few double-positive organoids were present (FIGS. 3F-G and 9D). These findings demonstrate that, once established in hTSCs, the allele-specific expression of MECP2 is maintained at the single organoid level. These clonal XCI dynamics are reminiscent of the patchy XCI pattern seen in the human placenta, which is believed to reflect the generation of villous trees from single trophoblast precursors.

Differentiation of SC-TOs into Invasive 3D EVT Organoids

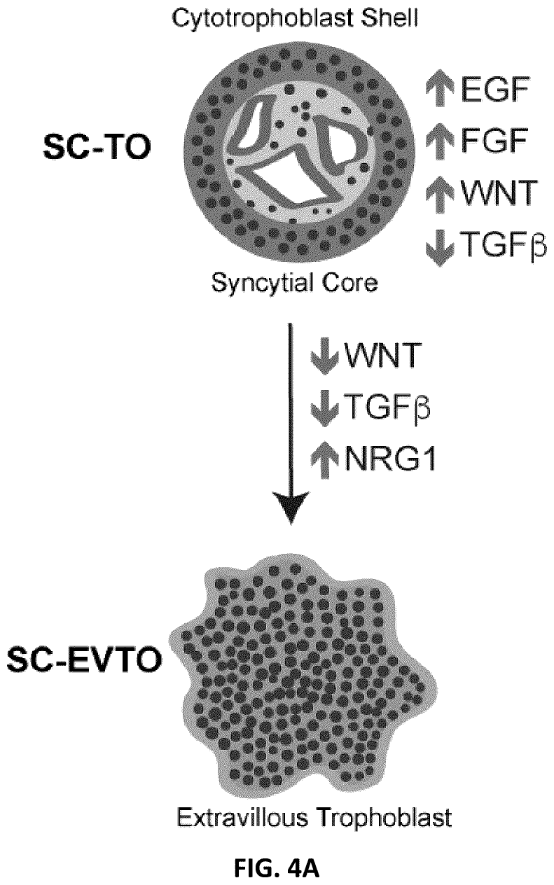

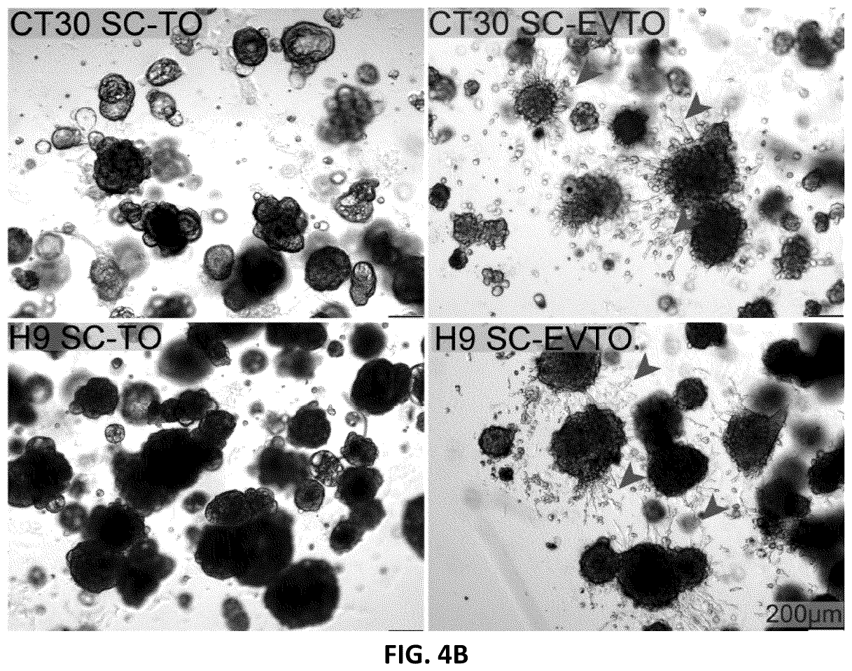

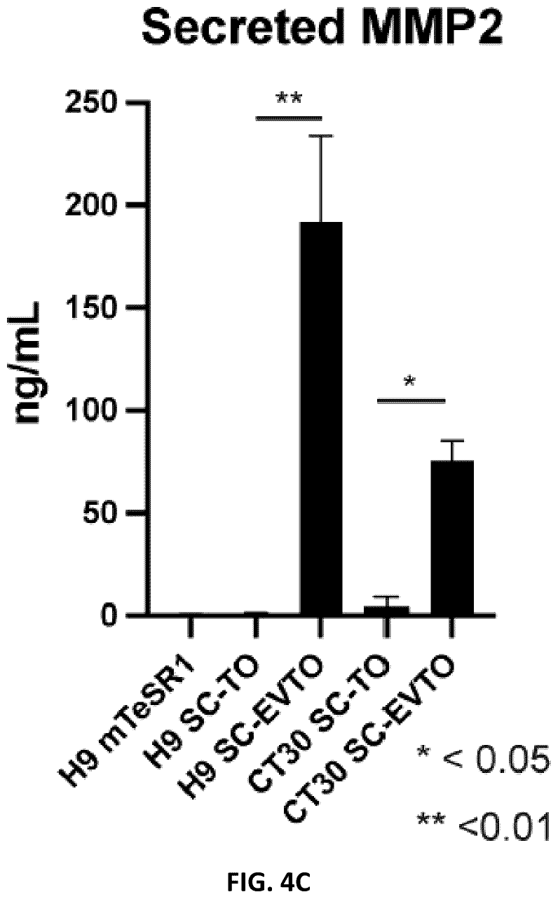

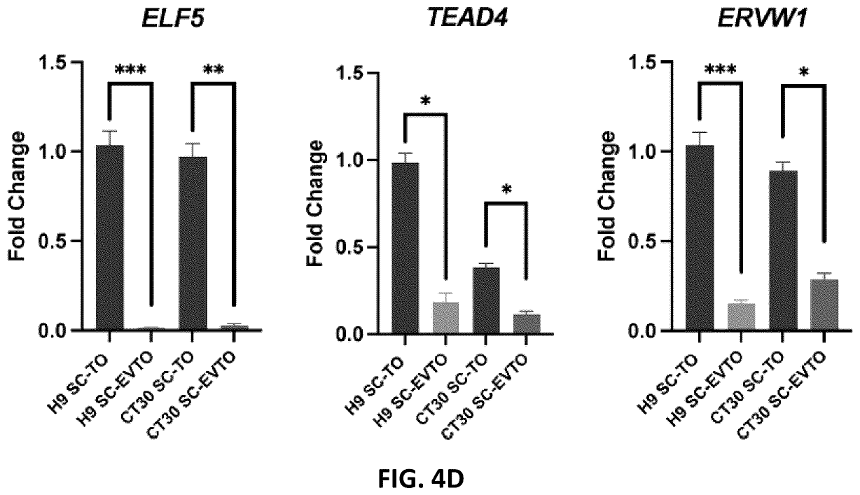

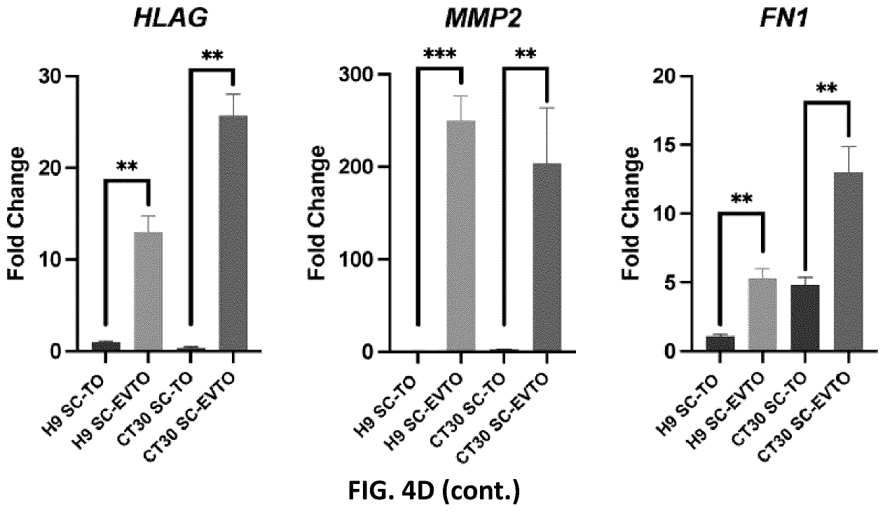

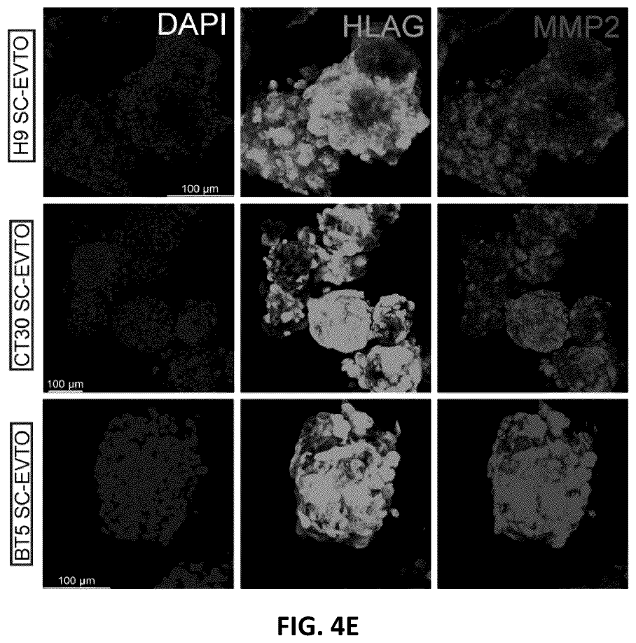

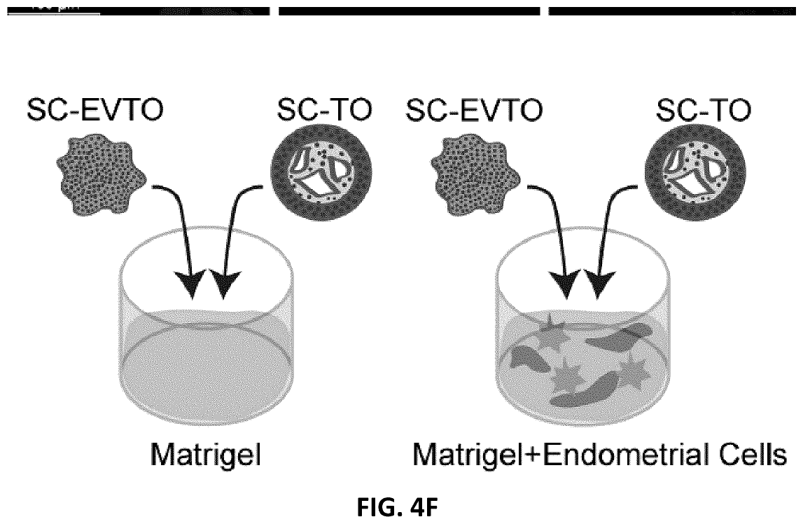

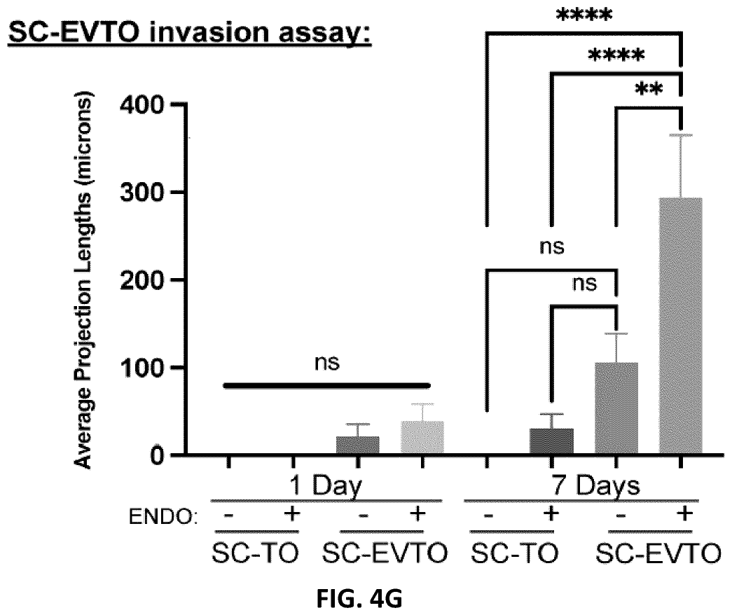

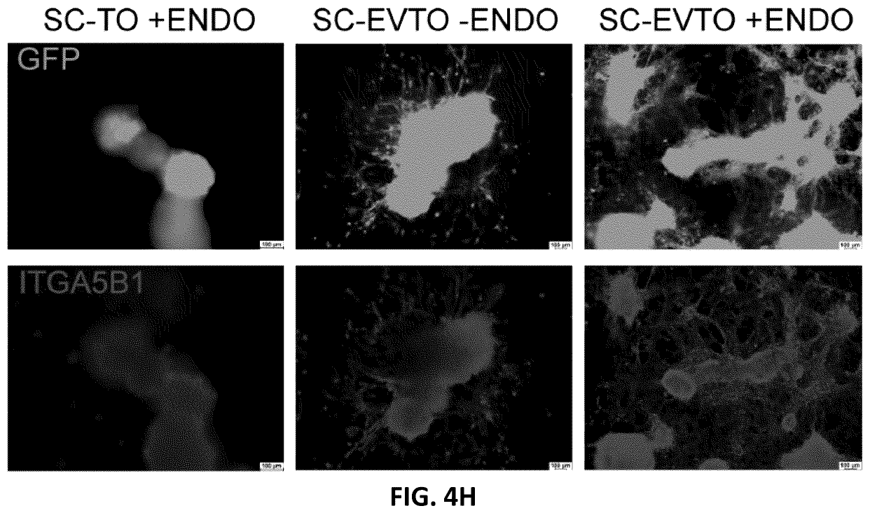

The above results indicate that the culture of naïve or primary hTSCs under standard TOM conditions yields organoids that largely contain CTBs with a syncytial core and only a small subset of primitive EVTs. Utilizing a previously reported methodology for differentiating primary trophoblast-derived organoids towards the EVT lineage, we were able to differentiate SC-TOs were able to be differentiated into specialized EVT organoids that exhibited a lack of cystic STB morphology and a prevalence of migratory cells (FIG. 4A-B, red arrows). These stem cell-derived EVT organoids (SC-EVTOs) displayed elevated secretion of MMP2 compared to SC-TOs (FIG. 4C). Gene expression in SC-TOs and SC-EVTOs generated from naïve and primary hTSC was compared by qRT-PCR. SC-EVTOs displayed reduced expression of the CTB markers ELF5 and TEAD4 and the STB marker ERVW1, but increased expression of EVT markers HLAG, MMP2, and FN1 (FIG. 4D). They also widely expressed HLAG and MMP2, markers of EVT differentiation and invasion, at the protein level (FIG. 4E). Overall, these results demonstrate that SC-TOs can undergo differentiation into specialized 3D EVT organoids.

A key characteristic of early placentation is the invasion of EVTs into the maternal decidua. Thus, the invasive potential of SC-TOs and SC-EVTOs was assayed in 3D Matrigel scaffolds in the presence or absence of human endometrial cells (FIG. 4F). For this purpose, Matrigel was combined with immortalized endometrial glandular cells and stromal fibroblasts. To facilitate the quantification of organoid projections, isogenic SC-TOs, and SC-EVTOs were labeled with a constitutively active lentiviral GFP vector. SC-TOs plated on Matrigel displayed few invasive projections over a seven-day period. In contrast, a substantial number of invasive projections emerged from SC-EVTOs in the presence of maternal endometrial cells (FIG. 4G). While SC-EVTOs in Matrigel alone formed some projections, significant projection length was only reached upon contact with maternal endometrial cells. This suggests that endometrial paracrine factors or extracellular matrix scaffolds formed by endometrial fibroblasts promote EVT invasion and maturity. These projections were positive for both the GFP reporter and the migratory EVT marker ITGA5B1 (FIG. 4H). It is concluded that SC-EVTOs, but not SC-TOs, have substantial invasive potential in the presence of maternal endometrial cells. The absence of invasive potential in SC-TOs may be attributed to the retention of epithelial gene expression signatures in the small primitive EVT population, as revealed by our single-cell transcriptome analysis (FIGS. 2A and 2C).

### 3D Trophoblast Organoids Display Selective Vulnerability to Emerging Viral Pathogens

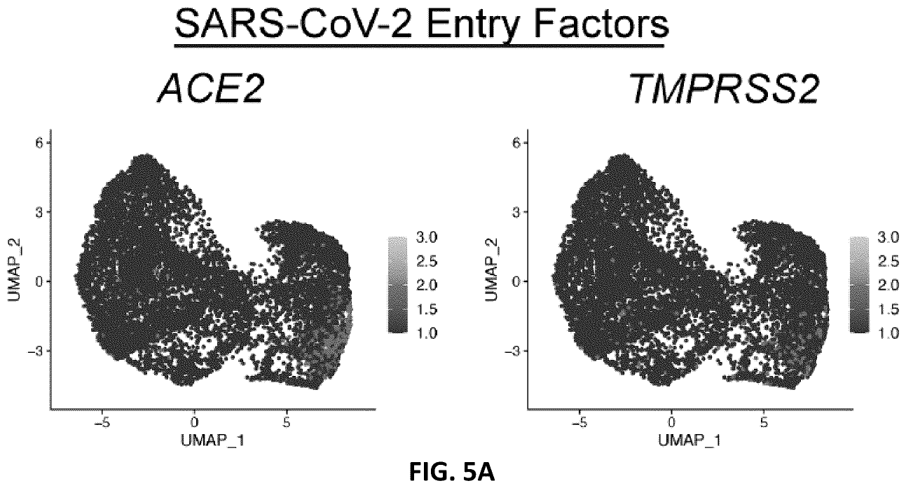

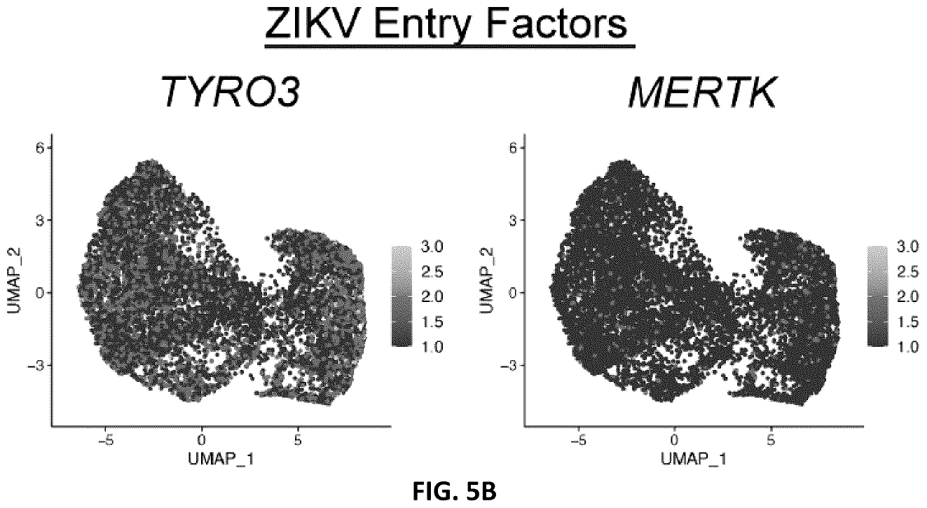

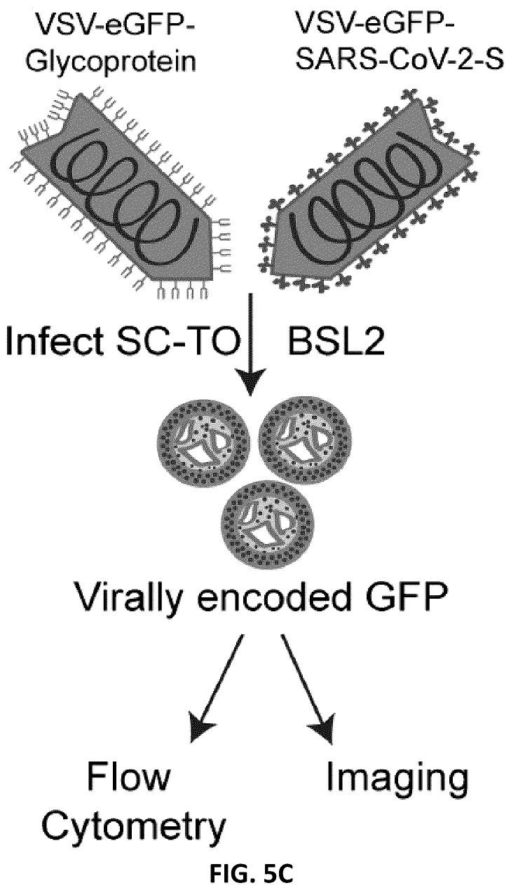

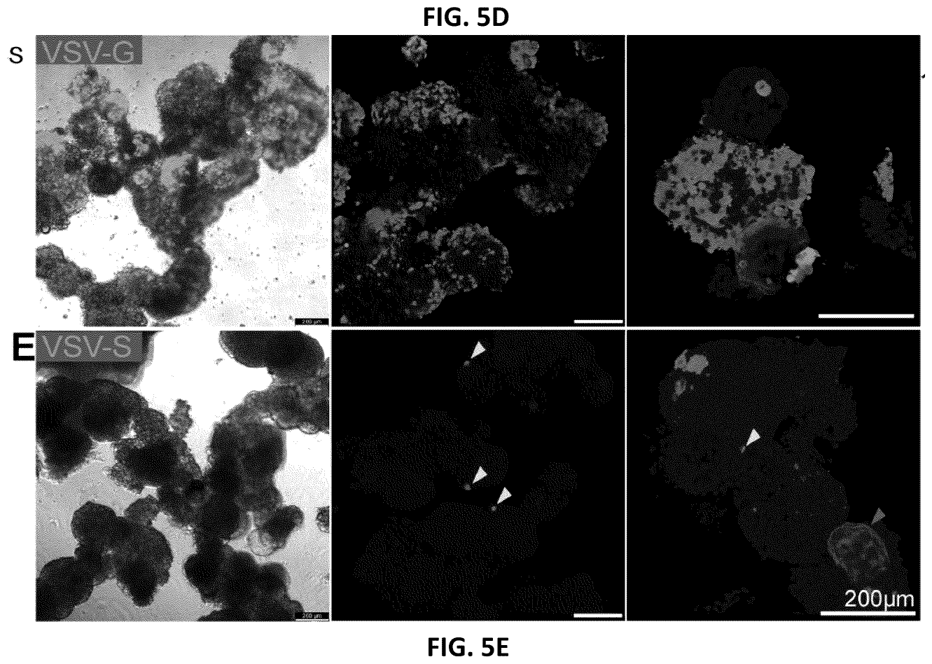

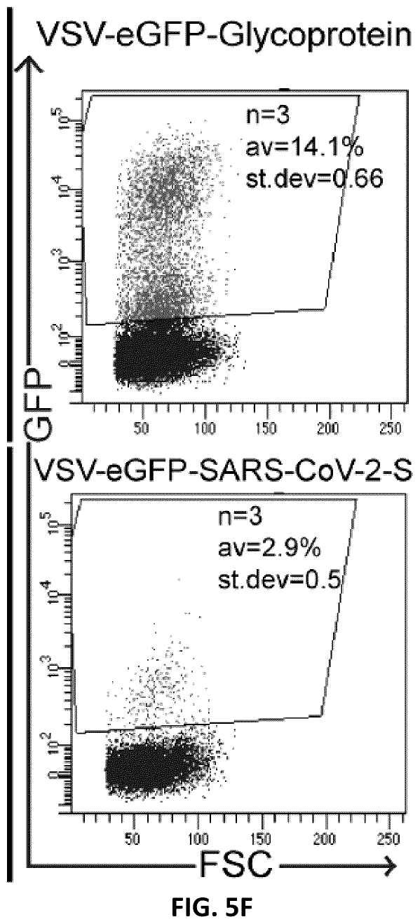

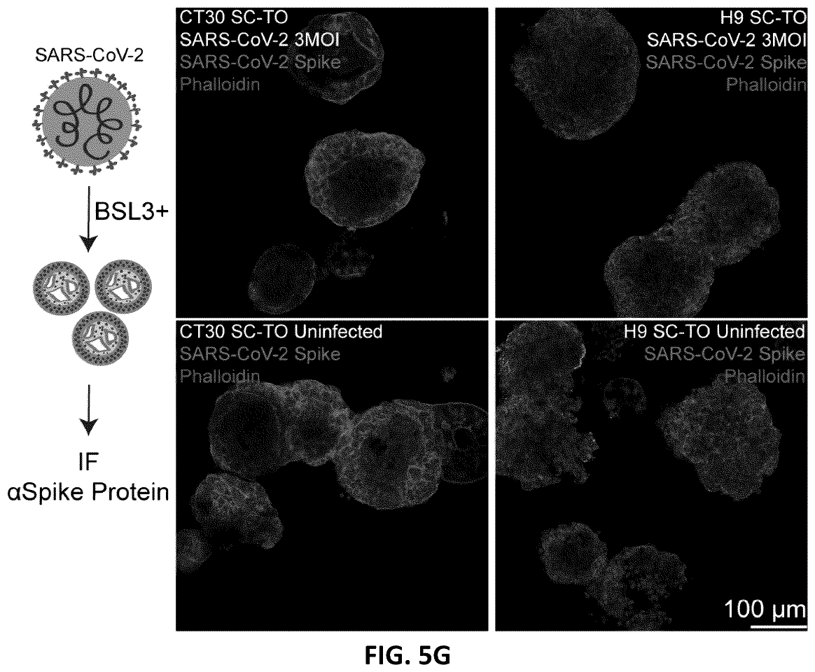

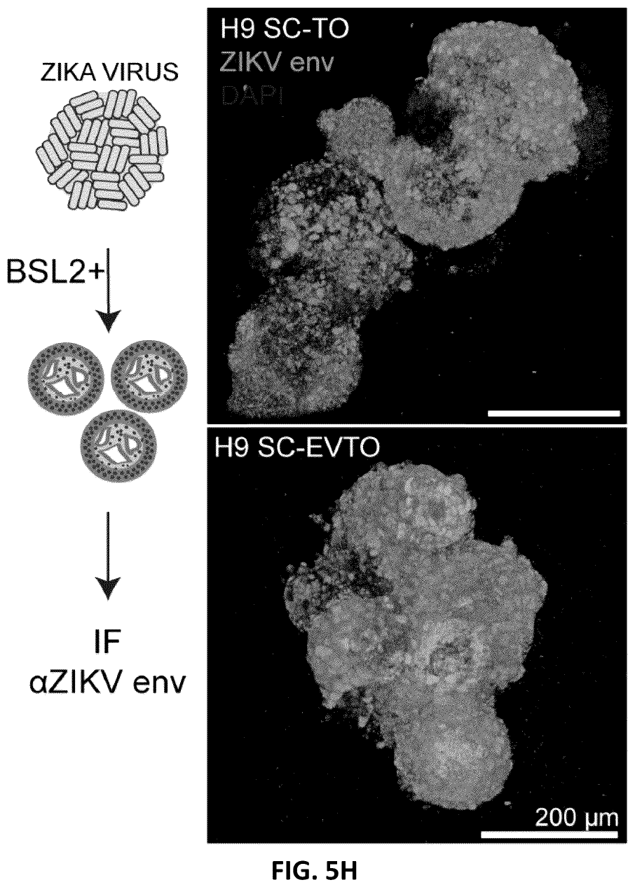

In addition to nurturing the developing fetus, the placenta serves as an important defense against infections. Of recent interest, the emerging viral pathogens SARS-COV-2 and ZIKV have been associated with adverse pregnancy outcomes, including preterm birth, still birth, and fetal/neonatal defects. Thus, whether SC-TOs can be used to study the permissiveness of early placental cells to these emerging pathogens was investigated. First, it was determined whether the SC-TOs express viral entry receptors for SARS-CoV-2 and ZIKV. The scRNA-seq data revealed limited co-expression of the SARS-CoV-2 viral entry receptors ACE2 and TMPRSS2 in the STB-2 cluster, which marks the mature STB subpopulation (FIG. 5A). In contrast, the ZIKV entry receptor TYRO3 was more widely expressed across the different clusters (FIG. 5B).

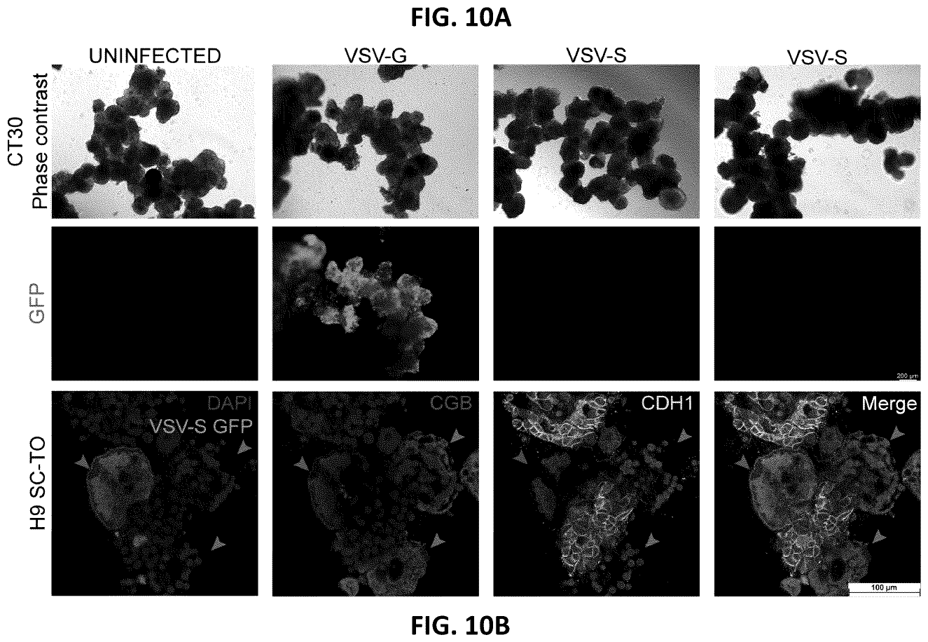

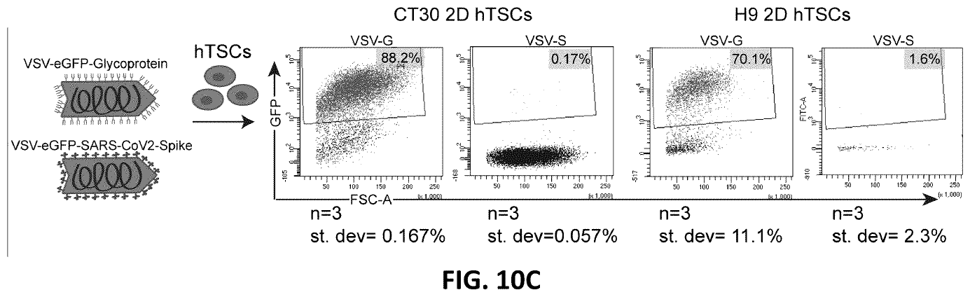

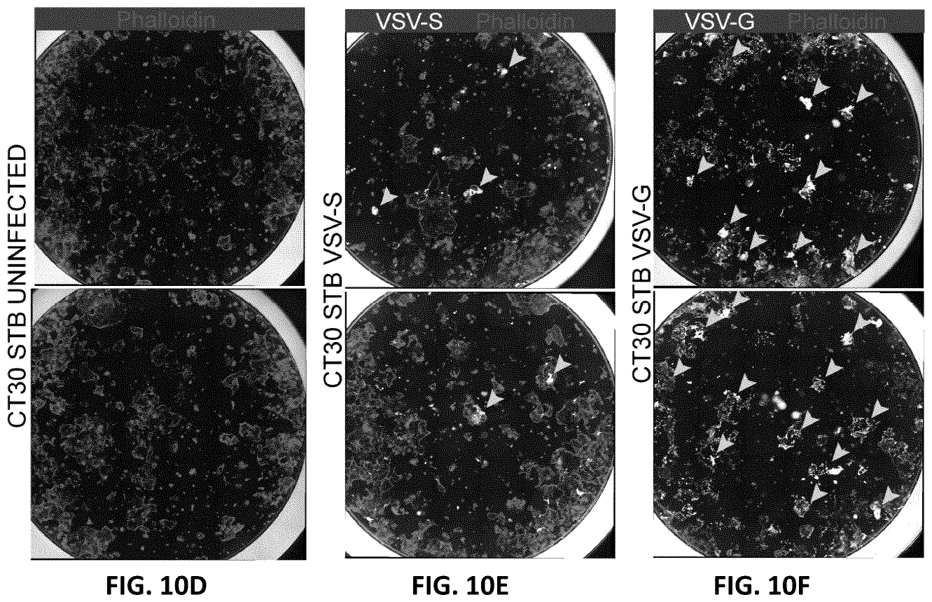

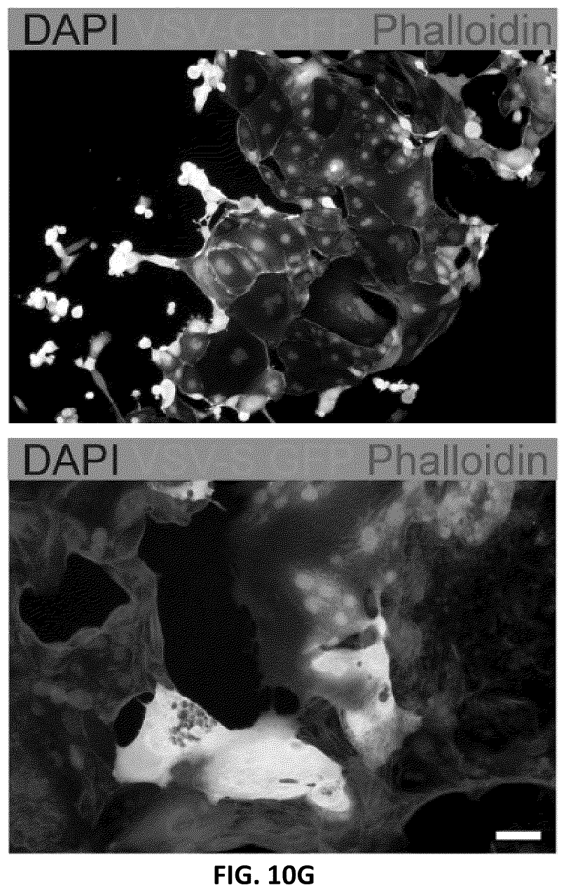

H9 and CT30 SC-TOs were infected with a SARS-CoV-2 pseudovirus comprising a replication-competent vesicular stomatitis virus (VSV) with the SARS-CoV-2 spike (S) protein, (VSV-S) (Case et al., 2020), which permits examination of SARS-CoV-2 entry, neutralization, and inhibition under reduced biosafety containment (FIG. 5C). The control VSV-eGFP-Glycoprotein (VSV-G) virus efficiently infected many cells within SC-TOs as revealed by fluorescent microscopy and flow cytometry (FIGS. 5D-F and 10A). In contrast, VSV-S infection was only detected in a few CTBs (FIG. 5E, yellow arrows) and a fraction of multinucleated STB-like cells (FIGS. 5E and 10B, red arrows). CT30 and H9 SC-TOs were also infected with a clinical isolate of SARS-CoV-2 under BSL3 conditions (FIG. 5G). IF analysis using an antibody specific to the SARS-CoV-2 Spike protein 24 h after infection revealed little signal compared to uninfected controls (FIG. 5G). In marked contrast, the Brazilian strain of ZIKV readily infected all cell types within SC-TOs (FIG. 5H), which is consistent with the widespread expression of the ZIKV entry receptor, TYRO3 (FIG. 5B). Collectively, these experiments indicate that SC-TOs display selective vulnerability towards SARS-CoV-2 and ZIKV.

The possibility that the inside-out architecture of 3D trophoblast organoids might limit viral access to the interior ACE2-expressing STB population was considered and therefore the findings were replicated in 2D hTSCs and their derivatives. Naïve and primary hTSCs were first infected with VSV-G/S and similar results were obtained as in SC-TOs: whereas hTSCs were highly susceptible to VSV-G infection, almost no infection was seen by VSV-S (FIG. 10C). VSV-G/S infections were also performed on STBs generated from 2D hTSCs in the presence of EGF, Forskolin, and ROCK inhibitor. While some of these 2D STBs were infected by VSV-S, a more robust infection was seen by VSV-G (FIG. 10D-G). It is concluded that mature STBs may be susceptible to infection by SARS-CoV-2, in accordance with their ACE2 expression profile (FIG. 5A), but early human trophoblast cells are more readily infected by VSV-G and ZIKV.

### DISCUSSION

The placenta has historically been considered the least understood human organ given the practical and ethical restrictions on studying its development in utero and the fact that animal models poorly recapitulate human placental physiology. In recent years two approaches have emerged to study trophoblast development in vitro: the isolation of 2D hTSCs from human blastocysts, first-trimester placental tissues, naïve hPSCs, or somatic cells and the derivation of 3D trophoblast organoids from first-trimester placental tissues. Here, it is shown that these two approaches can be combined to enable the isolation of self-renewing trophoblast organoids from naïve and primary hTSCs. These stem cell-derived trophoblast organoids (SC-TOs) display comparable tissue architecture, placental hormone secretion, and capacity for long-term self-renewal as primary trophoblast organoids. By performing in-depth single-cell transcriptome profiling, it is demonstrated that SC-TOs encompass a variety of trophoblast identities that closely correspond to CTB progenitor and differentiated cell states found in human post-implantation embryos. Furthermore, XCI dynamics were characterized during the establishment of SC-TOs from naïve hPSCs and it was demonstrated that SC-TOs can undergo lineage-specific differentiation into invasive 3D EVT organoids.

The single-cell transcriptomes of trophoblast organoids generated from naïve and primary hTSCs were remarkably similar, comprising five distinct trophoblast clusters in broadly comparable proportions. Given the divergent sources of these hTSC lines, this is an unexpected result: whereas primary hTSCs were isolated from first-trimester placental tissues, naïve hTSCs were obtained by reverting primed hESCs to naïve pluripotency and thereafter applying hTSC media. The relative scarcity of DEGs between the two SC-TO lines suggests that trophoblast organoid culture represents a powerful attractor state in which the influence of subtle epigenetic differences between naïve and primary hTSCs is mitigated. Furthermore, SC-TOs generated from naïve and primary hTSCs displayed comparable expression of most placenta-specific imprinted genes, despite the globally reduced levels of DNA methylation in naïve hPSCs and hTSCs. An important question for future research will be to determine whether these imprints are established through non-canonical epigenetic mechanisms, as was recently observed in mouse placental tissues.

Alignment with human trophoblast cell types in vivo indicated that SC-TOs contain a proliferative progenitor pool, CTB-1, which corresponds to CTBs found in the human post-implantation embryo. These proliferative CTBs maintain a stable transcriptome until at least 8 weeks of placental development. The pseudotime analysis indicates that trophoblast progenitors within CTB-1 bifurcate into either an EVT or STB trajectory. A second CTB cluster was observed that has exited the cell cycle and corresponds to non-proliferative CTBs and immature EVTs in vivo. This cluster likely represents a transitional population of CTBs undergoing differentiation towards the EVT lineage. While SC-TOs only contain a small subpopulation of primitive EVTs, the fraction of HLA-G and MMP2-expressing cells can be expanded by applying culture conditions that promote 3D EVT differentiation. SC-TOs also harbor two discrete clusters of STBs: a transitional cluster that resembles fusion-competent CTBs and a separate cluster of mature STBs that have exited the cell cycle and show upregulation of PSG and CGB family genes. Hence, SC-TOs reflect the cellular diversity of trophoblast identities found in the human post-implantation embryo, encompassing progenitor, transitional, and differentiated cell states.

Studies of human placental tissues at term have revealed the presence of large clonal patches in which either the maternal or paternal X chromosome is silenced. This patchy XCI pattern is thought to result from random XCI at an early stage of human trophoblast development, followed by the clonal expansion of single CTBs into villous trees. By using a double-color fluorescent reporter integrated into the X-linked MECP2 locus, it is shown that naive hESCs give rise to discrete populations of GFP-positive and tdTomato-positive hTSCs. The subsequent generation of SC-TOs resulted in the emergence of organoids that express a single fluorescent reporter allele, recapitulating clonal XCI patterns observed during human placental development. A recent study in non-human primate embryos reported that trophectoderm-derived extraembryonic cells undergo XCI soon after implantation, while XCI is more protracted in the embryonic lineages. It is proposed that the derivation of hTSCs from naïve hPSCs presents an experimentally tractable system in which to study the molecular events leading to XCI in the human trophoblast lineage. However, the extent to which XCI during trophoblast differentiation is truly random or skewed will require further investigation and may be influenced by the ability of specific naïve culture conditions to induce complete XCR.

In light of the evidence of increased rates of pregnancy complications in women who contracted SARS-CoV-2 (the virus that causes COVID-19) or ZIKV, whether early trophoblast cell types found within SC-TOs could recapitulate viral infectivity and be useful as models to study infection dynamics was examined. Consistent with the isolated expression of the entry factors ACE2 and TMPRSS2, a SARS-CoV-2 pseudovirus only infected a subset of STB-like cells. Furthermore, infection of SC-TOs with a clinical isolate of SARS-CoV-2 did not result in robust expression of the SARS-CoV-2 spike protein. These observations suggest that the SARS-CoV-2 virus may be capable of entering a fraction of STBs that express the appropriate receptors but cannot replicate efficiently once inside these cells. These findings contrast with the high rate of infection by ZIKV, which confirms prior reports that human first-trimester trophoblast cells are permissive to ZIKV infection and replication. Consequently, the first-trimester placenta may present a barrier to vertical transmission of SARS-CoV-2. The STB layer is shed and readily replaced during the first trimester and this turnover of susceptible cell types could provide additional protection from infection. In accordance with this interpretation, few consistent histopathological changes were observed in the placentas of SARS-CoV-2-positive women. These findings underscore that the placental barrier is functioning and possibly eliminating the virus prior to extensive damage to the placenta and fetus. It is hypothesized that the adverse pregnancy outcomes noted in women infected with SARS-CoV-2 could be a result of dysregulation of the renin-angiotensin system or immune responses. Another possibility is that comorbidities, such as vascular endothelial disruption caused by severe preeclampsia or obesity, may predispose the placenta to infection by SARS-CoV-2. This hypothesis could be investigated by generating SC-TOs from preeclamptic patients or by replicating the high-inflammatory environment seen in preeclampsia and re-examining the response to viral infection.

In summary, it is shown that self-renewing 3D trophoblast organoids can be isolated from naïve and primary hTSCs, which provides a methodology to model the impact of disease-associated mutations in a 3D microenvironment that reflects the cellular diversity of the first-trimester placenta. In agreement with the data, reduced expression of classical HLA molecules in trophoblast organoids has also been observed. This Example extends these findings by generating trophoblast organoids from naïve hPSCs and profiling their single-cell transcriptome, XCI status, and susceptibility to viral pathogens. The ability to genetically manipulate naïve hPSCs prior to differentiation into SC-TOs enables functional interrogation of regulatory factors implicated in placental organogenesis. Pertinent questions for future investigation will be to examine whether the long-term culture or in vivo transplantation of SC-TOs promotes maturation to later gestational stages, as has been described for other types of organoids, and to model placental vulnerability to other pathogens implicated in adverse pregnancy outcomes, such as cytomegalovirus and novel variants of SARS-CoV-2.

### Experimental Model and Subject Details

### Cell Lines and Culture Conditions

H9 (female) hESCs were obtained from the Washington University Genome Engineering and iPSC Center (GEiC), WIBR3 and WIBR3 MECP2-GFP/tdTomato hESCs (female) were obtained from Dr. Rudolf Jaenisch at the Whitehead Institute for Biomedical Research, BT5 (female), CT27 (female), and CT30 (female) hTSCs were obtained from Dr. William Pastor at McGill University with permission from Drs. Okae and Arima at Tohoku University, human endometrial stromal cells (female) were obtained from ATCC, and human endometrial epithelial cells (female) were 28 obtained from Dr. Pamela Pollock at Queensland University. The identities of the H9 and WIBR3 hESC lines used in this study were authenticated using Short Tandem Repeat (STR) profiling. The cell culture is regularly tested and negative for mycoplasma contamination. All experiments involving hESCs were approved by the Institutional Biological and Chemical Safety Committee and Embryonic Stem Cell Research Oversight Committee at Washington University School of Medicine. Details about the culture conditions for each cell line are provided below.

Culture of Primed and Naïve hPSCs

Primed hPSCs were cultured in mTeSR1 Plus (STEMCELL Technologies, #100-0274) on hESC-qualified Matrigel (Corning, 354277) coated wells and passaged using ReLeSR (STEMCELL Technologies, 05872) every 4 to 6 days. Primed hPSCs were cultured at 37 degrees Celcius in 5% CO2 and 20% O₂. Naive hPSCs were cultured on mitomycin C-inactivated mouse embryonic fibroblast (MEF) feeder cells and were passaged by a brief PBS wash followed by a 5-minute incubation in TrypLE Express (Gibco, 12604) to disperse the cells to single-cells. Centrifugation occurs in fibroblast medium [DMEM (Millipore Sigma, #SLM-021-B) supplemented with 10% FBS (Cytiva, SH30088.03), 1× GlutaMAX (Gibco, 35050), and 1% penicillin-streptomycin (Gibco, 15140)]. Naive hPSCs were cultured in the 5i/L/A media. 500 mL of 5i/L/A was generated by combining: 240 mL DMEM/F12 (Gibco, 11320), 240 mL Neurobasal (Gibco, 21103), 5 mL N2 100× supplement (Gibco, 17502), 10 mL B27 50× supplement (Gibco, 17504), 10 μg recombinant human LIF (PeproTech, 300-05), 1× GlutaMAX, 1×MEM NEAA (Gibco, 11140), 0.1 mM β-mercaptoethanol (Millipore Sigma, 8.05740), 1% penicillin-streptomycin, 50 μg/ml BSA Fraction V (Gibco, 15260), and the following small molecules and cytokines: 1 μM PD0325901 (Stemgent, 04-0006), 1 μM IM-12 (Enzo, BML-WN102), 0.5 μM SB590885 (Tocris, 2650), 1 μM WH4-023 (A Chemtek, H620061), 10 μM Y-27632 (Stemgent, 04-0012), and 10 ng/mL Activin A (Peprotech, 120-14). Naïve hPSCs were cultured in 5% 02, 5% CO2 at 37° C. For primed to naïve hPSC conversion, 2×10⁵ single primed cells were seeded on a 6-well plate with an MEF feeder layer in 2 mL mTeSR1 supplemented with 10 μM Y-27632. Two days later, the medium was switched to 5i/L/A. After 7 to 10 days from seeding, the cells were expanded polyclonally by using TrypLE Express on a MEF feeder layer. Tissue culture media were filtered using a 0.22 μm filter. Media were changed every 1-2 days. Naïve hPSCs before passage 10 were used for experiments.

The WIBR3-MECP2 reporter cell line was converted to naïve pluripotency using the alternative PXGGY/A naïve induction cocktail. Primed WIBR3-29 MECP2 cells were maintained in mTeSR1 Plus as described above, dissociated with TrypLE Express, and 200,000 single cells were plated upon mitomycin C inactivated MEFs. For the first 48 hours, cells were maintained in mTeSR1 Plus with 10 uM Y-27632, then switched to PXGGY/A medium for 10-12 days. ROCK inhibitor Y-27632 was only added for 24 h after passaging. The culture was passaged 2 times every 3-4 days followed by flow cytometry analysis and hTSC derivation.

hTSC Culture

Collagen IV (5 μg/mL) was used as the substrate and was coated overnight at 37° C. in 6-well plates. Medium contained DMEM/F12 supplemented with 0.1 mM 2-mercaptoethanol, 0.2% FBS, 0.5% Penicillin-Streptomycin, 0.3% BSA, 1% ITS-X (Gibco, 51500), 1.5 μg/ml L-ascorbic acid (Wako, 013-12061), 50 ng/ml EGF (Peprotech, AF-100-15), 2 μM CHIR99021 (R&D #4423), 0.5 μM A83-01 (Peprotech, 90943360), 1 μM SB431542 (BioVision, 1674), 0.8 mM VPA (Sigma Aldrich, P4543), and 5 μM Y-27632 and was changed daily. Cells were passaged with TrypLE every 3-4 days and 50,000 cells were passaged for continued growth. All experiments were performed using hTSCs between 20-30 passages. Derivation of hTSCs from naïve hPSCs was performed as previously described.

### Derivation of Stem Cell-Derived Trophoblast Organoids (SC-TOs)

2D hTSCs were grown in hTSC medium until cells reached 80% confluency. hTSCs were single-cell dissociated with TrypLE and washed twice in Advanced DMEM/F12. 3,000 cells were suspended in 30 uL Matrigel droplets to a final concentration of 72% Matrigel in Advanced DMEM/F12. Droplets were seeded in 24 well plates. A two-minute incubation step on the benchtop was required before turning the plate over and polymerizing the Matrigel droplets at 37° C. for 30 minutes. Plates were subsequently removed from the incubator and 500 uL of trophoblast organoid medium (TOM) was added. TOM medium was prepared as described previously [Advanced DMEM/F12 (Life Technologies #12634010), 1X N2 Supplement (Life Technologies #17502048), 1X B27 Supplement minus vitamin A (Life Technologies 12587010), 100 ug/mL Primocin (Invivogen #ant-pm-1), 1.25 mM N-Acetyl-L-Cysteine (Sigma #A9165), 2 mM L-Glutamine (Life Technologies #25030-024), 500 nM A83-01 (Peprotech #9094360), 1.5 uM CHIR99021 (Stemgent #04-0004), 2 uM Y-27632 (Stemgent #04-0012), 50 ng/mL rhEGF (Peprotech #AF-100-15), 50 ng/mL rhHGF (Peprotech #100-39), 80 ng/mL rhR-Spondin1 (Peprotech #120-38), 100 ng/mL rhFGF2 (Peprotech #100-18B), and 2.5 uM Prostaglandin E2 (Millipore Sigma #P0409)]. 500 uL of TOM was changed daily and organoids were maintained for 8-10 days between passages. Matrigel droplets were washed once with PBS and 1 mL of TrypLE Express was used to break up droplets and dissociate the organoids for 20 minutes at 37° C. in a 1.5 mL Eppendorf tube, which was inverted several times throughout the incubation. Following incubation, tubes were washed three times in Advanced DMEM/F12 by centrifugation at 1000 rpm for 3-5 minutes. Between each wash, 100 uL of Advanced DMEM/12 was added and organoids were dissociated by trituration approximately 20 times or until a single-cell suspension of organoids was obtained. A 40 μm filter was employed to remove large, unbroken chunks of organoids. 3,000-5,000 cells were re-seeded per 30 μL Matrigel droplet.

### Method Details

Differentiation of 2D hTSCs

hTSCs were grown to 80% confluency in hTSC medium and split by using TrypLE Express into a single-cell suspension. These cells were resuspended in EVT medium-1 (DMEM/F12 supplemented with 0.1 mM 2-mercaptoethanol, 0.5% Penicillin-Streptomycin, 0.3% BSA, 1% ITS-X supplement, 100 ng/ml NRG1, 7.5 μM A83-01, 2.5 μM Y27632, and 4% KnockOut Serum Replacement) and 75,000 cells per well were plated in 1 ug/mL of Collagen IV pre-coated wells. Matrigel was added to the medium at a final concentration of 0.5%. EVT cells were allowed to grow in this medium for 3 days. Following this, the medium was changed to EVT medium-2, which lacks NRG1 but is otherwise the same as EVTm-1, and Matrigel was added to this medium to a final concentration of 0.5%. On day 6, the cells were split into new Collagen IV-coated wells with TrypLE Express at a 1:2 ratio into EVT medium-3, which is the same as EVTm-2 but lacks KOSR. Matrigel was once again added at 0.5% and EVTs were allowed 2 additional days of growth.

STB differentiation from hTSCs in adherent culture was performed as previously described with minor modifications. 3.75×10⁴ hTSCs were added to 24 well plates coated with 2.5 ug/mL Collagen IV. hTSCs were resuspended in STB medium (DMEM/F12 supplemented with 0.1 mM 2-mercaptoethanol, 0.5% Penicillin-Streptomycin, 0.3% BSA, 1% ITS-X supplement, 2.5 mM Y27632, 50 ng/ml EGF, 2 mM forskolin, and 4% KSR) and allowed to grow for 3 days. At day 3, an equal proportion of STB medium was added to wells and incubated for another 3 days. Infection with VSV-G and VSV-S occurred on day 5 for 24 hours as described below.

Differentiation of SC-TOs into Specialized 3D EVT Organoids

Like 2D EVT differentiation, EVT organoids were differentiated by using phases of EVT media. 3,000-5,000 dissociated organoids were seeded in 30 uL Matrigel droplets in 24 well plates as described above and cultured in TOM media for 3 days. Media were changed to organoid EVT medium-1 [Adv. DMEM/F12, 0.1 2-mercaptoethanol, 0.5% Penicillin/Streptomycin, 0.3% bovine serum albumin, 1% ITS-X, 100 ng/mL NRG1, 7.5 uM A83-01, and 4% knockout serum replacement] for 5 days and 500 uL-1 mL of media were replaced each day per well. Subsequently, organoid EVT medium-2 was applied, which is identical to organoid EVTm-1 except for the omission of NRG1. EVTm-2 was replaced for an additional 2-3 days, or until robust migratory cells sprouted from the organoids.

### Human Endometrial Invasion Assay

Immortalized human endometrial stromal cells and immortalized human endometrial epithelial cells were both maintained in phenol-red free DMEM/F12 supplemented with 7.5% charcoal-stripped fetal bovine serum, 1× non-essential amino acids, 1× Insulin/Transferrin/Selenium, and 1× Antibiotic-Antimycotic at 37° C. and 5% CO2. Endometrial epithelial cells were nucleofected (Lonza BioScience) with a pBRY-nuclear mCherry plasmid (Addgene #52409), and stably transfected cells were obtained by FACS, sorting for mCherry fluorescence on three consecutive passages (98%+ cells were red at last sorting). For the 3D cultures, each well of an 8-well chamber slide was loaded with 100 uL ice-cold Matrigel containing 4×10⁵ human endometrial fibroblasts. After Matrigel was solidified for 30 minutes at 37° C., 200 uL culture medium containing 2.5×10⁴ nuclear-mCherry endometrial epithelial cells were seeded on top of the Matrigel. Control wells contained Matrigel and culture medium without cells. The chamber slides were incubated overnight before SC-TO/SC-EVTO seeding. H9 hTSCs were lentivirally transduced with a constitutively active FUW-GFP virus, and high GFP-expressing cells were obtained by FACS. These GFP-labeled H9 hTSCs were used to derive SC-TOs by transfer to TOM medium for 8 days or SC-EVTOs by transfer to TOM medium for 3 days, followed by 5 days of EVT organoid medium. Organoids were removed from the Matrigel droplets by incubating them in Cell Recovery solution on ice for 30 minutes. Whole organoids were resuspended in an endometrium medium containing Estradiol (36 nM), Medroxyprogesterone 17-Acetate (1 μM), and c-AMP (100 μM) and seeded upon the endometrial-matrigel surface with medium changes every other day. Organoids attached to the endometrial-matrigel surface overnight were fixed and analyzed for GFP-labeled organoid projection lengths after 7 days of co-culture using ImageJ software.

Movies were taken on a Zeiss Cell Discoverer 7 confocal microscope equipped with 5% CO₂ and 37° C. incubation temperature. GFP labeled H9 SC-TOs and SC-EVTOs upon endometrial interface (epithelial cells labeled mCherry) were imaged for 21 hours on day 5 of interaction.

### Immunofluorescence

Organoids were isolated from Matrigel droplets by trituration using a 1000 μl pipette tip along the bottom of the well in Cell Recovery Solution (Corning #354253). Cell Recovery Solution with Matrigel and organoids in suspension was collected in a 1.5 mL tube and placed on ice for 30 minutes. Organoids were gently centrifuged (600 rcf for 5 min) and washed in PBS once before fixation in 4% Paraformaldehyde on ice for 30 minutes. Fixed organoids were washed with PBS+0.1% BSA 3 times prior to permeabilization and blocking overnight at 4° C. in PBS+4% BSA+5% FBS+0.5% Triton-X. Primary antibodies were added in staining solution (PBS+4% BSA+5% FBS+0.1% Tween-20) and incubated overnight at 4° C. Secondary antibodies were incubated overnight at 4° C. in a staining solution at a concentration of 1:300. Phalloidin-670 and Hoechst dyes were also added at this step. Organoids were mounted in Prolong Gold Antifade mounting medium (ThermoFisher #P36930) between two coverslips separated by Grace Bio-Labs Cover Well incubation chambers (Grace Bio-Labs #645401). Confocal imaging was performed on a Leica SP8 Single photon confocal microscope. Widefield imaging was performed on a Leica DMi-8 fluorescence microscope.

### Light-Sheet Microscopy

Organoids were immunostained as described above and then cleared in a Glycerol-Fructose clearing solution (60% glycerol and 2.5M fructose). Cleared organoids were resuspended and embedded in 50% clearing solution and 50% water+2.5% low melting point agarose. SC-TOs in agarose were drawn into a light sheet microscope capillary tube and allowed to solidify overnight at 4° C. before imaging the next day on a Zeiss Lattice Lightsheet 7 Microscope. CDH1, SDC1, and DAPI signals were analyzed for volume (microns³) using Amira software.

### Flow Cytometry

Flow cytometry was performed to analyze the proportion of cells expressing GFP fluorescence as a result of VSV-eGFP-SARS-CoV-2-S/G infection. Cells grown in 2D (hTSCs, 2D EVT) were infected for 1 hour and the medium was changed to a non-infectious medium for 8 hours. Cells were dissociated with TrypLE Express and fixed in 4% PFA in suspension for 10 minutes. Cells were washed three times with PBS+0.1% BSA. Cells were passed through a 40-micron cell strainer and run on a BD LSRFortessa X-20 and analyzed by using FlowJo software. Uninfected control samples were used as a negative GFP signal and compared to VSV-G (positive GFP control) and VSV-S (experimental samples).

### RNA Isolation and Quantitative RT-PCR

Total RNA was isolated using the E.Z.N.A. total RNA kit I (Omega, D6834), and cDNA synthesis was performed on total RNA using the high-capacity cDNA reverse transcription kit (Applied Biosystems, 4368814). Real-time PCR was performed using PowerUp SYBR Green master mix (Applied Biosystems, A25743) on the StepOnePlus Real-Time PCR System (Applied Biosystems). All analyses were done in triplicate among 2-3 biological replicates. Gene expression was normalized to RPLP0. Error bars represent the standard deviation (SD) of biological replicate fold change values.

For miRNA analysis, total RNAs, including small RNAs were isolated using the Qiagen miRNeasy mini kit. cDNA synthesis was performed using the TaqMan Advanced MicroRNA cDNA Synthesis Kit, followed by TaqMan probe real-time PCR. We followed all the manufacturer's specified instructions. The following thermocycling conditions were used for qRT-PCR: 95° C. for 20 sec, 40 cycles of 95° C. for 1 sec, and 60° C. for 20 sec. miRNA expression was normalized to a ubiquitously expressed miRNA, hsa-mir-16-5p. All analysis was done in technical triplicates. Error bars represent the standard deviation of 2-3 biological replicates.

### hCG and MMP2 ELISAs

hTSCs were plated at 30,000 cells per Matrigel droplet and 10 days later the medium was collected. The medium was collected from organoid droplet wells after 24 hours of incubation for all experiments. Dilution factors were first determined on test samples before final experimentation. The manufacturer's instructions were followed in the execution of the ELISAs.

### Single-cell RNA Sequencing

Single-cell suspensions for scRNA-seq were prepared by dissociating organoids in Papain enzyme solution (MP Biomedicals, 100921). Organoids were removed from Matrigel droplets in cell recovery solution on ice for 20 minutes. Free organoids were washed once in ice-cold HBSS and centrifuged at 4000 RPM for 30 seconds. The supernatant was removed and 400 μL of activated Papain solution was added. Organoids were incubated at 37° C. for 10 minutes, inverting the tube every 2 minutes. 600 μL of 10% FBS in DMEM with 5 mM magnesium was added to each sample. Gentle trituration of the sample was performed 3-5 times with a P1000 pipet. Following this, 10 μl of DNase I was added to each tube and inverted gently to mix. Samples were next incubated at 37° C. for 5 minutes and gently triturated an additional 3-5 times. Organoids were centrifuged at 4000 rpm for 30 seconds. The supernatant was removed, dissociated organoids were resuspended in PBS+0.04% BSA, and the cell suspension was passed through a 40-micron filter. Trypan blue was used to test viability (>90% live cells).

cDNA was prepared after the GEM generation and barcoding, followed by the GEM-RT reaction and bead cleanup steps. Purified cDNA was amplified for 11-13 cycles before being cleaned up using SPRIselect beads. Samples were then run on a Bioanalyzer to determine the cDNA concentration. GEX libraries were prepared as recommended by the 10× Genomics Chromium Single-cell 3′ Reagent Kits (v3 Chemistry) user guide with appropriate modifications to the PCR cycles based on the calculated cDNA concentration. For sample preparation on the 10× Genomics platform, the Chromium Single-cell 3′ GEM, Library and Gel Bead Kit v3 (PN-1000075), Chromium Single-cell B Chip Kit (PN-1000153), and Dual Index Kit TT Set A (PN-1000215) were used. The concentration of each library was accurately determined through qPCR utilizing the KAPA library Quantification Kit according to the manufacturer's protocol (KAPA Biosystems/Roche) to produce cluster counts appropriate for the Illumina NovaSeq6000 instrument. Normalized libraries were sequenced on a NovaSeq6000 S4 Flow Cell using the XP workflow and a 28×10×10×150 sequencing recipe according to manufacturer protocol. A median sequencing depth of 50,000 reads/cell was targeted for each Gene Expression Library.

### VSV-eGFP-SARS-CoV-2-S, VSV-G, and ZIKV Infection in Trophoblast Organoids

The chimeric pseudovirus, VSV-eGFP-SARS-CoV-2-S, was kindly provided by Dr. Sean P. J. Whelan, Washington University School of Medicine in Saint Louis. The chimeric virus was synthesized by integrating the SARS-CoV-2 Spike gene (Wuhan-Hu-1 isolate) in an infectious 35 molecular clone of Vesicular stomatitis virus (VSV). VSV-eGFP-SARS-CoV-2-S and VSV-eGFP-G were propagated in Vero cells, and titration of the virus was performed using the qPCR standard curve described previously. The Brazilian strain of ZIKV was provided by S. Whitehead (Bethesda, MD) and obtained initially from P. F. C. Vasconcelos (Instituto Evandro Chagas, Levilândia, Brazil). ZIKV was propagated in Vero cells, and the titer was calculated by focus forming assay (FFU) as described previously. Trophoblast organoids were infected with VSV-eGFP-SARS-CoV-2-S and VSV-eGFP-G, at 5, and 1 multiplicity of infection (MOI), respectively, and incubated for 24 h at 37° C., 5% CO₂, and 70% relative humidity. Monolayer trophoblasts were incubated with 5 or 1 MOI of VSV-S or VSV-G, respectively, for one hour. Subsequently, the medium was changed to non-infectious medium overnight and analyzed the following day. ZIKV infection was performed at 0.1 MOI for 2 h at 37° C., 5% CO₂, and 70% relative humidity. After 2 h of ZIKV infection, the media were removed, organoids were washed with PBS, and fresh media was added and further incubated for 24 h. After incubation, the culture supernatant was harvested, and organoids were fixed using 1% PFA. All experiments were performed under biosafety level 2 (BSL2) conditions.

### SARS-CoV-2 Infection

H9 and CT30 organoids were counted at 10 days of growth and seeded at 3.8×10⁵ and 8×10⁴ cells per well in 500 μL of DMEM supplemented with 2% FBS, 10 mM HEPES, penicillin and streptomycin (D2F) in a 24-well tissue culture plate. These cells were infected with a multiplicity of infections of 30, 3, and 0.3 of the WA1 strain of SARS-CoV-2 for one hour at 37° C. This equates to 11.4×10⁶, 11.4×10⁵, and 11.4×10⁴ pfu and 2.4×10⁶, 2.4×10⁵, and 2.4×10⁴ pfu for the H9 and CT30 cells respectively. This virus was expanded on Vero cells overexpressing human TMPRSS2 virus and sequenced by next-generation sequencing to confirm the identity and presence of the Furin-cleavage site in the Spike protein. Next, the cells were transferred gently to 15 mL conical tubes and spun for 5 minutes at 200×g. The supernatant was aspirated, and the cells were resuspended in 1.0 mL of D2F and transferred to a fresh 24-well tissue culture plate. 24 h hours later, the cells were collected in a 15 mL conical tube and spun for 5 minutes at 200×g. Following removal of the tissue culture supernatant, the cells were washed gently in PBS+0.1% BSA and spun again for 5 minutes at 37° C. After removing the supernatant, the cells were fixed with 500 μL 4% paraformaldehyde for 30 minutes at 20° C. Next, the fixed cells were spun for 5 minutes at 200×g and washed with 3 mL of PBS+0.1% BSA, followed by a final centrifugation for 5 minutes at 200×g. Following the removal of the supernatant, the cells were resuspended in 1.0 mL PBS+0.1% BSA before further analysis.

### Quantification and Statistical Analysis

### ELISA Data Analysis

GraphPad Prism was used to create visual graphs and analyses. Statistical significance was determined by a one-way ANOVA with Sidak's multiple comparisons tests. Significance was determined by p<0.05. *P value<0.05, **P value<0.01.

qRT-PCR Data Analysis

Fold change values were determined by 2\hat{ }-ddCt and means among 2-3 replicates were visualized in each graph. Statistical analysis was determined using the dCt values of each analyzed group, and a paired t-test or one-way ANOVA was used to determine statistical significance. Ns=not significant, or p>0.05, *=p<0.05, **=p<0.01, ***=p<0.001, and ****=p<0.0001.

scRNA-seq Analysis

Raw sequencing data were converted to fastq format using the cellranger mkfastq command (v.5.0.0). scRNA-seq reads were aligned to the GRCh38 (UCSC hg38) reference genome and quantified using the cellranger count command using default parameters.

Count data were processed using the R package Seurat (v.4.0.0), using Gencode v.31 for gene identification and considering only protein-coding genes. Cells with less than 2,500 or more than 8,000 informative genes expressed, cells with less than 15,000 sequenced fragments, and cells with more than 25% mitochondrial gene content were excluded. Count data were log-normalized and scaled to 10,000. scRNA-seq data sets for H9 ESC-derived TSC organoids, CT30-derived TSC organoids, and replicates respectively, were transformed and mitochondrial gene content was regressed out using SCTransform as implemented in Seurat. The data sets were integrated using 4,000 anchors following SC transformation.

PCA analysis was based on the 3,500 most variable genes. Nearest neighbors were computed using the top 6 principal components, and 5 clusters were identified using the Louvain community detection implemented in Seurat's FindClusters function (‘resolution=0.3’). 2-dimensional representations were generated using uniform manifold approximation and projection (UMAP). For each cluster, the genes differentially expressed between H9 ESC and CT30-derived organoids were determined using the FindMarkers function and a Wilcoxon Rank Sum test (‘log fc.threshold=0.1, min.pct=0.25’). GO term enrichment analyses were performed with the clusterProfiler package in R. To infer developmental trajectories, quality-filtered count data were normalized, and replicates were integrated using the ingest function in the Python package Scanpy (v.1.2.8; scanpy.readthedocs.io). Diffusion maps were calculated with destiny as implemented in Scanpy.

To compare with publicly available scRNA-seq data sets, raw counts were obtained from GEO (accession: GSE89497 (Liu et al., 2018); GSE136447 (Xiang et al., 2020)), and re-analyzed as described above using SC transformation. For the Liu et al. data set, the markers provided as a Supplementary Table in the paper were used to reproduce and reassign the cell types to clusters; for the Xiang et al. data set, cell types were directly provided as Supplementary Table. Processed and annotated public scRNA-seq data sets were integrated with our combined TSC organoid scRNA-seq data sets using reciprocal PCA as implemented in Seurat.

### Allele-Specific Analysis of X-Linked Gene Expression

scRNA-seq reads of H9-derived organoids were aligned to chrX of the GRCh38 (UCSC hg38) reference genome with cellranger count command, and reads for each single cell were extracted from the sam file guided by the barcodes as listed in the “CB:Z” column by cellranger. Heterozygous SNPs for the H9 (p25) stem cell line were obtained from an Affymetrix genome-wide SNP 6.0 Array on GEO (accession: GSE15096). 5 nt sequence windows in exons, +/−2 nt around the SNP, were extracted from the alignment files for each single cell using the GenomicAlignments package in R; SNPs that were covered by at least 50 reads over all single cells were considered for further analysis. The alignment quality of the reads was inspected manually by using BLAT as implemented on the UCSC browser. Some SNPs (rs10521478, rs14115, rs8575, rs7392258) that were covered by multi-mapping reads only were discarded. For each single cell, the number of reads with the reference allele or alternate allele was counted using custom PERL scripts. Bulk RNA-seq data sets for primed and naïve H9 hESCs derived in 5i/L/A were obtained from GEO (accession: GSE138688 (Dong et al., 2020)), and aligned with HISAT2 (http://daehwankimlab.github.io/hisat2) to the GRCh38 (UCSC hg38) reference genome. Read alignments on SNPs were extracted with the GenomicAlignments package in R and allele frequencies were analyzed as described for SNPs that were covered by at least 10 reads in naïve hESCs.

Key Resources Table

| | | |
|---|---|---|
| REAGENT | SOURCE | IDENTIFIER |

Antibodies

| | | |
|---|---|---|
| anti-KRT7 | Dako/Agilent | M7018 (RRID: AB_2134589) |
| anti-TP63 | Abcam | ab124762 (RRID: AB_10971840) |
| anti-SDC1 | Abcam | ab34164 (RRID: AB_778207) |
| anti-E-Cadherin | Cell Signaling | 3195S (RRID: AB_2291471) |
| (rabbit) | | |
| anti-E-Cadherin | Cell Signaling | 14472S (RRID: AB_2728770) |
| (mouse) | | |
| anti-hCG beta | Abcam | ab53087 (RRID: AB_870731) |
| anti-MMP2 | Cell Signaling | 40994S (RRID: AB_2799191) |
| anti-hCG alpha | R&D | MAB4169 (RRID: AB_2079126) |
| anti-HLAG | Santa Cruz | sc-21799 (RRID: AB_627938) |
| Pan HLA-A, B, C | Biolegend | 311413 (RRID: AB_493133) |
| (W6/32-488) | | |
| anti-ZIKV capsid | GeneTex | GTX133317 (RRID: AB_2756861) |
| anti-Ki67 | Cell Signaling | 9449S (RRID: AB_2797703) |
| anti-SARS-CoV-2 | Gift from Dr. Ellebedy, | Clone number 1C02 |
| Spike protein | Washington University | |
| | in St. Louis | |
| anti-ITGA5B1 | Abcam | ab275977 |
| (rabbit) | | |
| Secondary ab-Donkey | Thermo Fisher | A21202 (RRID: AB_141607) |
| anti-mouse 488 | Scientific | |
| Secondary ab-Donkey | Thermo Fisher | A21206 (RRID: AB_2535792) |
| anti-rabbit 488 | Scientific | |
| Secondary ab-Donkey | Thermo Fisher | A31570 (RRID: AB_2536180) |
| anti-mouse 555 | Scientific | |
| Secondary ab-Donkey | Thermo Fisher | A31572 (RRID: AB_162543) |
| anti-rabbit 555 | Scientific | |
| Secondary ab-Donkey | Thermo Fisher | SA5-10126 (RRID: AB_2556706) |
| anti-human 488 | Scientific | |

Chemicals, peptides, and recombinant proteins

| | | |
|---|---|---|
| Phalloidin-670 | Cytoskeleton | PHDN1 |
| CHIR99021 | R&D | 4423 |
| A83-01 | Peprotech | 9094360 |
| SB431542 | BioVision | 1674 |
| Valproic Acid | Sigma-Aldrich | P4543 |
| Insulin-Transferrin- | Gibco | 51500056 |
| Selenium-Ethanolamine | | |
| (ITS -X) | | |
| Insulin-Transferrin- | Gibco | 41400045 |
| Selenium (ITS -G) | | |
| Forskolin | Sigma-Aldrich | F3917 |
| Y-27632 | Stemgent | 04-0012 |
| PD0325901 | Stemgent | 04-0006 |
| IM-12 | Enzo | BML-WN102 |
| SB590885 | Tocris | 2650 |
| WH-4-023 | A Chemtek | S1180 |
| XAV939 | Sigma | X3004 |
| Gö6983 | Tocris | 2285 |
| GDC-0994 | Selleck | S7554 |
| | Chemicals | |
| rhEGF | Peprotech | AF-100-15 |
| rhHGF | Peprotech | 100-39 |
| rhR-Spondin1 | Peprotech | 120-38 |
| rhFGF2 | Peprotech | 100-18B |
| rhNRG1 | Cell Signaling | 5218SC |
| Activin A | PeproTech | 120-14 |
| LIF | PeproTech | 300-05 |
| Prostaglandin E2 | Millipore Sigma | P0409 |
| hESC qualified | Corning | 354277 |
| Matrigel | | |
| Dispase | Stem Cell | 07923 |
| | Technologies | |
| TrypLE Express | Gibco | 12604-013 |
| Primocin | Invitrogen | ant-pm-1 |
| N-Acetyl-L-Cysteine | Sigma Aldrich | A9165 |
| Knock-out Serum | ThermoFisher | 10828028 |
| Replacement | | |
| Collagen IV | Corning | 354233 |
| Papain | MP Biomedicals | 100921 |
| Phenol-red free | Sigma Aldrich | D-2906 |
| DMEM/F12 | | |
| Charcoal-stripped | Thermo | A3382101 |
| fetal bovine serum | Scientific | |
| Ibidi micro well | Ibidi | 80826 |
| chamber slides | | |
| Medroxyprogesterone | Sigma-Aldrich | M1629 |
| 17-acetate | | |
| beta-Estradiol | Sigma-Aldrich | E8875 |
| 8-Bromo-cAMP | Tocris | 1140 |

Critical commercial assays

| | | |
|---|---|---|
| E.Z.N.A. total | Omega | D6834 |
| RNA kit | | |
| miRNeasy Mini Kit | Qiagen | 1038703 |
| TaqMan Advanced | Applied | A28007 |
| miRNA cDNA | Biosystems | |
| Synthesis Kit | | |
| TaqMan Fast | Applied | 4444556 |
| Advanced Master | Biosystems | |
| Mix | | |
| DNeasy Blood and | Qiagen | 69504 |
| Tissue Kit | | |
| MMP2 ELISA Kit | Abcam | ab100606 |
| hCG ELISA Kit | CalBiotech | HC251F |

Deposited data

| | | |
|---|---|---|
| Raw and Processed | This paper | GEO: GSE172241 |
| data | | |
| A developmental | Xiang et al., 2020 | GEO: GSE136447 |
| landscape of | | |
| 3D-cultured human | | |
| pre-gastrulation embryos | | |
| Single-cell RNA-seq | Liu et al., 2018 | GEO: GSE89497 |
| reveals the diversity | | |
| of trophoblast subtypes | | |
| and patterns of | | |
| differentiation in the | | |
| human placenta | | |

Experimental models: Cell lines

| | | |
|---|---|---|
| H9 (WA09); female | WashU GEiC | RRID: CVCL_9773 |
| WIBR3 (hESC); female | Whitehead | RRID: CVCL_9767 |
| | Institute | |
| WIBR3-MECP2-GFP/ | Whitehead | N/A |
| tdTomato; female | Institute | |
| bTS5 (BT5) hTSC; female | Drs. Okae, Arima, | RCB Cat# RCB4940 (RRID: CVCL_A6JH) |
| | and Pastor | |
| CT27 hTSC: female | Drs. Okae, Arima, | RCB Cat# RCB4936 (RRID: CVCL_A7AZ) |
| | and Pastor | |
| CT30 hTSC; female | Drs. Okae, Arima, | RCB Cat# RCB4938 (RRID: CVCL_A7BB) |
| | and Pastor | |
| Human Endometrial | ATCC | CRL-4003 (RRID: CVCL_C464) |
| Stromal Cells- | | |
| Immortalized | | |
| Human endometrial | Dr. Pamela Pollock, | N/A |
| epithelial cells | Queensland Un. of | |
| (EM-TERTs)-Immortalized | Technology, Australia | |

MicroRNA probes

| | | |
|---|---|---|
| TaqMan MicroRNA | Applied | A25576 |
| Advanced Assay | Biosystems | |
| 477860_mir | | |
| Assay Name: hsa-miR-16-5p | | |
| TaqMan MicroRNA | Applied | A25576 |
| Advanced Assay | Biosystems | |
| 478703_mir | | |
| Assay Name: miR-1323 | | |
| TaqMan MicroRNA | Applied | A25576 |
| Advanced Assay | Biosystems | |
| 479396_mir | | |
| Assay Name: miR-525-5p | | |
| TaqMan MicroRNA | Applied | A25576 |
| Advanced Assay | Biosystems | |
| 479485_mir | | |
| Assay Name: miR-517a-3p | | |
| TaqMan MicroRNA | Applied | A25576 |
| Advanced Assay | Biosystems | |
| 478148 mir | | |
| Assay Name: miR-518b | | |
| TaqMan MicroRNA | Applied | A25576 |
| Advanced Assay | Biosystems | |
| 478986_mir | | |
| Assay Name: miR-519d-3p | | |

Software and algorithms

| | | |
|---|---|---|
| FlowJo_v10.6.2 | FlowJo ™ | RRID: SCR_008520; https://www.flowjo.com/ |
| Prism 9 | GraphPad | RRID: SCR_002798; https://www.graphpad.com |
| FIJI | NIH | https://imagei.net/Fiji |
| Amira | ThermoFisher | N/A |
| R 4.0.0 | R project | N/A; https://www.r-project.org/ |
| Cellranger 5.0.0 | 10Xgenomics | https://support.10xgenomics.com/single-cell-gene-expression/software/downloads/latest |
| ClusterProfiler 4.0.0 | Bioconductor | https://bioconductor.org/packages/release/bioc/html/clusterProfiler.html |
| Seurat 4.0 | Satija Lab | https://satijalab.org/seurat |
| pheatmap 1.0.12 | CRAN | https://cran.r-project.org/web/packages/pheatmap/index.html |

What is claimed is:

1. A model system of human placental development comprising a 3D stem cell-derived trophoblast organoid (SC-TO) comprising self-organized trophoblast stem cells, wherein the trophoblast stem cells are isolated from naïve human pluripotent stem cells.

2. The system of claim 1, wherein the self-organized trophoblast stem cells of the SC-TO comprise a portion of cytotrophoblast cells positioned at a periphery of the SC-TO and a portion of multinucleated syncytiotrophoblast cells positioned within an interior of the SC-TO.

3. The system of claim 2, wherein the self-organized trophoblast stem cells of the SC-TO further comprise a portion of primitive extravillous trophoblasts.

4. The system of claim 1 wherein the SC-TO displays clonal X chromosome inactivation patterns.

5. The system of claim 1, wherein the SC-TO exhibits vulnerability to a pathogen selected from SARS-CoV-2 or Zika virus.

6. The system of claim 5, wherein the vulnerability is correlated with an expression level of entry factors of the pathogens

7. The system of claim 6, wherein the entry factors comprise at least one of ACE2, TMPRSS2, and TYRO3.

8. The system of claim 1, further comprising a 3D extravillous trophoblast organoid comprising a plurality of extravillous trophoblasts produced by differentiating the self-organized trophoblast stem cells into the extravillous trophoblasts.

9. A method of generating a stem cell-derived trophoblast organoid (SC-TO), the organoid comprising self-organized trophoblast stem cells isolated from naïve human pluripotent stem cells, the method comprising isolating naive human pluripotent stem cells from human samples, seeding the naïve human pluripotent stem cells in a 3D scaffold, and culturing the naïve human pluripotent stem cells in a TSC culture composition.

10. The method of claim 9, wherein the self-organized trophoblast stem cells of the SC-TO comprise a portion of cytotrophoblast cells positioned at a periphery of the SC-TO and a portion of multinucleated syncytiotrophoblast cells positioned within an interior of the SC-TO.

11. The method of claim 9, wherein the self-organized trophoblast stem cells of the SC-TO further comprise a portion of primitive extravillous trophoblasts.

12. The method of claim 9, wherein the SC-TO displays clonal X chromosome inactivation patterns.

13. The method of claim 7, wherein the SC-TO exhibits vulnerability to a pathogen selected from SARS-CoV-2 or Zika virus.

14. The method of claim 10, wherein the vulnerability is correlated with an expression level of entry factors of the pathogens.

15. The method of claim 6, wherein the entry factors comprise at least one of ACE2, TMPRSS2, and TYRO3.

16. A method of screening for molecules as a treatment for a pregnancy-related disease, complication, or disorder, the method comprising:
a. inducing the pregnancy-related disease, complication, or disorder in a 3D model system of human placental development, the system comprising a 3D stem cell-derived trophoblast organoid (SC-TO) comprising self-organized trophoblast stem cells isolated from naïve human pluripotent stem cells; and
b. administering a candidate molecule to the SC-TO and selecting the candidate molecule as a treatment if the candidate molecule reduces the pregnancy-related disease, complication, or disorder in the SC-TO.

17. The method of claim 16, wherein the pregnancy-related disease, complication, or disorder comprises a viral infection.

18. The method of claim 16, wherein the viral infection comprises an infection by a pathogen selected from a SARS-CoV-2 virus and a Zika virus.

19. The method of claim 18, wherein the at least a portion of the self-organized trophoblast stem cells of the SC-TO expresses at least one entry factor of the pathogen.

20. The method of claim 19, wherein the at least one entry factor is selected from ACE2, TMPRSS2, and TYRO3.
